(19) United States
(12) Patent Application Publication
(10) Pub. No.: US 2026/0191405 A1
(43) Pub. Date: Jul. 9, 2026

(54) INTRAORAL SCANNER AND SMART TOOTHBRUSH FOR ORAL HEALTH

(71) Applicant: ALIGN TECHNOLOGY, INC., San Jose, CA (US)

(72) Inventors: Sindhuja SHOLAVARAM, Andhra Pradesh (IN); Gajula Anjani Prabhu SANKEERTH, Hyderabad (IN); Ritvik KHARE, Madhya Pradesh (IN); Sukanya SINGH, Patna (IN); Safiuz Zama KHAN, Uttar Pradesh (IN); Chaitanya VADDISRIRAM, Hyderabad (IN); Sai Lokesh CHEEKATI, Telangana (IN)

(21) Appl. No.: 19/433,538

(22) Filed: Dec. 26, 2025

Related U.S. Application Data
(60) Provisional application No. 63/742,282, filed on Jan. 6, 2025.

(51) Int. Cl. A61B 1/24; A61B 1/00; A61B 1/05; A61B 5/00; A61B 5/06; A61C 17/22; A61C 17/32

(52) U.S. Cl. CPC A61B 1/24; A61B 1/00009; A61B 1/05; A61B 5/065; A61B 5/4547; A61B 5/4552; A61B 5/6887; A61C 17/221; A61C 17/32; A61B 2560/0462

(57) ABSTRACT

A method for using intraoral scan data to guide patient toothbrushing may include receiving brushing guidance and a 3D model of a patient's dentition, activating the toothbrush to start a tooth brushing activity, determining, during toothbrushing activity, a location of the toothbrush with respect to the patient's dentition based on data from sensors on the toothbrush and the 3D model, correlating the toothbrush location with the brushing guidance, and adjusting an operation of the toothbrush based on the correlation of the toothbrush location with the brushing guidance.

### RELATED APPLICATIONS

This application claims the benefit under 35 U.S.C. § 119(e) of U.S. Provisional Ser. No. 63/742,282 , filed Jan. 6, 2025, and titled “Intraoral Scanner and Smart Toothbrush for Oral Health,” which is incorporated herein, in its entirety, by this reference.

### BACKGROUND

Proper oral hygiene is useful in maintaining overall oral health and well-being. Common oral diseases include dental caries (cavities), periodontal diseases, gum recession, and other oral health problems, which can significantly impact an individual's quality of life. Despite its importance, many individuals struggle with maintaining optimal oral hygiene. This struggle often stems from two main issues: a lack of proper oral hygiene knowledge and the use of poor oral hygiene techniques.

The maintenance of oral hygiene typically involves mechanical plaque removal using toothbrushes, interproximal cleaning devices, such as floss, and mouthwashes. Despite the availability of these tools, patient adherence to recommended oral hygiene practices remains suboptimal. Many patients lack adequate knowledge about the best practices for oral hygiene. This includes not only the frequency and duration of brushing and flossing but also the correct methods to perform these tasks effectively. There is often a significant gap in understanding the correct technique for brushing and flossing, which can greatly influence the effectiveness of these practices. This lack of knowledge can lead to ineffective oral hygiene routines that fail to prevent dental health issues.

Furthermore, even when patients are informed about proper oral hygiene practices, they often struggle with the application of these techniques. Poor brushing techniques, such as incorrect brush angles, inadequate brushing time, and improper flossing methods, are common. These suboptimal practices can prevent the effective removal of plaque, thereby increasing the risk of oral diseases. Additionally, many individuals do not use dental aids such as dental floss, water picks, and mouthwash as effectively as they could, further diminishing their oral health.

Many patients inadequately care for hard to reach areas such as the posterior teeth, interdental or interproximal spaces, and subgingival regions. Thes areas often receive inadequate cleaning which contributes to the accumulation of plaque and the development of oral diseases.

Furthermore, the motivation to adhere to daily oral hygiene routines is often low, especially in younger demographics, leading to irregular and inadequate cleaning habits.

The consequences of inadequate oral hygiene are profound, contributing to both short and long-term dental problems, which can lead to pain, discomfort, and even systemic health issues.

The present disclosure provides systems and methods that address the limitations of current oral hygiene practices and tools, simplify the process of cleaning, increase the effectiveness of plaque removal, enhance accessibility to difficult-to-reach areas, and ultimately improve patient compliance and oral health outcomes.

### SUMMARY

Embodiments of the present disclosure provide improved systems and methods for using intraoral scanning systems and smart toothbrushes to improve a patient's oral health. For example, the systems and methods described herein may integrate intraoral scanning data with smart toothbrush data to improve a patient's oral health.

### BRIEF DESCRIPTION OF THE DRAWINGS

A better understanding of the features, advantages and principles of the present disclosure will be obtained by reference to the following detailed description that sets forth illustrative embodiments, and the accompanying drawings of which:

FIG. 1 depicts an intraoral scanning system, in accordance with some embodiments;

FIG. 2 shows intraoral scan data generated using an intraoral scanning system, in accordance with some embodiments;

FIG. 3 shows a toothbrushing system that uses scan data of the intraoral scanning system, in accordance with some embodiments;

FIG. 4 shows smart toothbrush data generated by the toothbrushing system, in accordance with some embodiments;

FIG. 5 shows toothbrush guidance data, in accordance with some embodiments;

FIG. 6 shows a method of generating guidance using an intraoral scanning system, in accordance with some embodiments;

FIG. 7 shows a method of brushing teeth using guidance from an intraoral scanning system, in accordance with some embodiments;

FIG. 8 shows a method of providing real-time feedback and guidance based on intraoral scanning data and smart-toothbrushing data, in accordance with some embodiments;

FIG. 9 shows toothbrushing data generated using a smart toothbrush, in accordance with some embodiments;

FIG. 10 shows toothbrushing data generated using a smart toothbrush, in accordance with some embodiments; and

FIG. 11 shows a method of using smart toothbrush data and intraoral scanning data to generate and provide dental health guidance, in accordance with some embodiments.

### DETAILED DESCRIPTION

The following detailed description provides a better understanding of the features and advantages of the inventions described in the present disclosure in accordance with the embodiments disclosed herein. Although the detailed description includes many specific embodiments, these are provided by way of example only and should not be construed as limiting the scope of the inventions disclosed herein.

The methods, apparatus, and systems disclosed herein are well suited for combination with prior devices such as intraoral scanners, for example the iTero system commercially available from Align Technology, Inc.

The presently disclosed methods and systems are well suited for combination with prior approaches to scanning intraoral structures and smart toothbrushes to improve oral heath by providing feedback to dentists and patients on brushing techniques and potential or developing oral health problems and to educate and motivate patients to have healthy oral care habits.

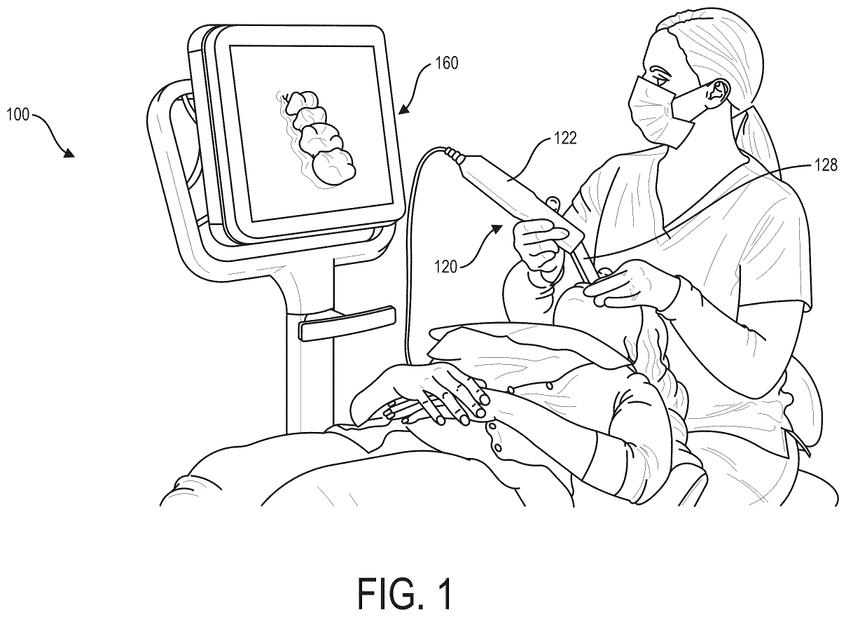

Reference is now made to FIG. 1 which depicts an intraoral scanning system 100, in accordance with some embodiments of the present disclosure. The intraoral scanning system 100 comprises an elongate handheld wand 120 that has a probe 128 at distal end of the handheld wand 120. As used herein, the proximal end of the handheld wand is the end of the handheld wand that is closest to a user's hand when the user is holding the handheld wand in a ready-for-use position and the distal end of the handheld wand is the end of the handheld wand that is farthest from the user's hand when the user is holding the handheld wand in a ready-for-use position.

In some embodiments, an intraoral scanner may include an intraoral imaging system including an image sensor and a light projector, such as a structured light projector, or other 3D imaging system, disposed in proximal end of probe 122.

Although an embodiment of the intraoral 3D scanner is provided in FIG. 1 many different types of 3D scanning hardware and software may be used. For example, the 3D scanning system may be a confocal 3D scanning system, a photogrammetry scanner, or other 3D scanning system type.

A structured light projector includes a light source. In some applications, structured light projector focuses light from light source at a projector focal plane that may be located external to the probe and at an object to be scanned. Structured light projector may have a pattern generator that is disposed in the optical path between light source and the projector focal plane. Pattern generator generates a structured light pattern at projector focal plane when light source is activated to transmit light through pattern generator.

The scanning system may include an imaging sensor comprising an array of pixels, e.g., a CMOS image sensor. The intraoral scanner 100 may include control circuitry that controls the operation of the light sources and the image sensor to capture images of a patient's dentition. Using information from the intraoral scanner, a computer processor may reconstruct a three-dimensional image of the surface of object, such as a patient's dentition.

In general, these methods and apparatuses may be configured so that the intraoral scanning system includes a wand (having one or more cameras, one or more light sources, etc.). The one or more cameras may include a camera sensor(e.g., CCD CMOS sensor) and optics (e.g., beam splitters, filters, gratings, polarizers, wavelength selective quarter waveplate, etc.) that may be configured to detect near-IR, fluorescence, visible light (e.g., white light), and/or single-wavelength light (e.g., red, blue, green, etc.). Any of these apparatuses may include one or more light sources to emit one or more wavelengths appropriate for near-IR, white light, single-wavelength light, etc. Any of these apparatuses may be configured to emit light that results in fluorescence in the teeth or other oral targets. In some cases the intraoral scanner includes a housing (e.g., base, cabinet, etc.) that encloses all or some of one or more processors and associated circuitry, including memory. For example, these apparatuses may include a base or control system 160 enclosing the one or more processors and the memory. The intraoral scanning systems described herein may include one or more visible light sources on the wand (e.g., near-IR light source, such as an LED, white light source, such as an LED, laser, etc.).

During the scanning process, as the scanner is manually maneuvered throughout the mouth, the scanner captures a multitude of images from various angles.

As the scanner captures images, point cloud data is generated from each of the images. Each point within these clouds represents a specific location on the surface of the dentition, encoded in three-dimensional coordinates. These points are derived from the reflections and distortions of the scanner's light as it interacts with the dental surfaces, which the scanner's sensors and cameras capture and convert into digital data sets of point clouds.

The point clouds are stitched together. The stitching process may involve the alignment and integration of multiple point clouds to assemble a three-dimensional model of the oral cavity including, for example 3D models of the upper and lower dentition. The process includes detecting similar 3D structures in overlapping portions of the point clouds and aligning the point clouds with each other based on these overlapping areas.

The 3D digital model, derived from the intraoral scanner's data, can be used in various dental applications, including for example, orthodontic, restorative, and other dental treatment planning processes, the creation of orthodontic aligners, dental crowns, and bridges, the assessment of oral health, and other processes.

During the scanning process, 2D images of the patient's oral cavity, such as the dentition, including the teeth, and the gums, may be captured. The 2D images may include 2D surface images and 2D subsurface images, such as those captured with near-IR illumination.

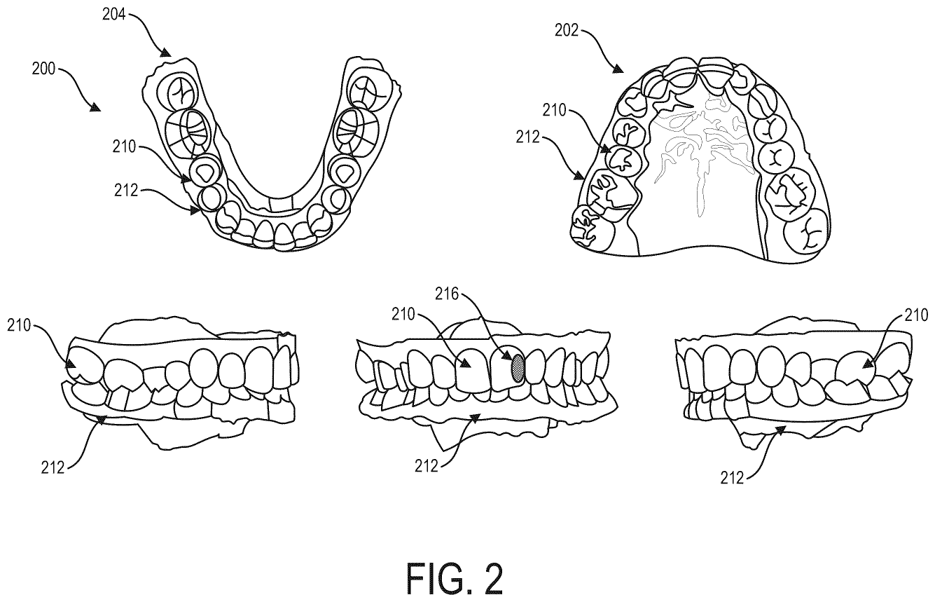

FIG. 2 depicts a 3D model 200 of a patient's dentition. The 3D model may include models of the upper arch 202 and the lower arch 204. The model 200 may include the patent's teeth 210 and the gingiva 212. The model may include information including the location of the gingival margin.

In some embodiments, the intraoral scanner may capture and/or detect locations of dental defects 216, such as tooth decay, plaque buildup and/or dental caries (cavities), such as by using a plaque or caries stain before scanning the teeth.

Stains may be used to detect dental caries (also referred to as cavities). A caries dye or disclosing solutions may selectively adhere to areas of demineralization. Demineralization is an early stage of tooth decay. These areas have altered chemical composition compared to healthy enamel or dentin. The stain or disclosing solutions may be configured to bind to the areas of demineralization more readily or more strongly than to areas of health enamel or dentin. The staining agent is applied directly to the teeth. After the application, the teeth may be scanned with a 3D intraoral scanner. The areas of decay absorb the stain and appear differently colored compared to the healthy enamel in the scan data. For instance, a common color for these stains is red or blue, which provides a sharp contrast to the natural color of the tooth, making the carious lesions more visible. The 3D scan may capture 3D and color data for the teeth with the dyed or stained locations on the teeth indicating potential locations of caries.

Similar to detection of caries, stains may also be used to detect dental plague. The process involves using a disclosing agent, such as a dye or stain that is designed to selectively adhere to plaque, with little to no adherence to the clean surfaces of teeth. The dye binds to the plaque deposits, staining them a vivid color (commonly pink, red, or blue) that contrasts sharply with the natural color of teeth, making the plaque clearly visible.

After the application of the disclosing agent, the teeth may be scanned with a 3D intraoral scanner. The areas of with plaque buildup absorb the stain and appear differently colored compared to the cleaned portions of the teeth which provides a sharp contrast to the natural color of the tooth, making the plaque more visible. The 3D scan may capture 3D and color data for the teeth with the dyed or stained locations on the teeth indicating potential locations of dental plaque buildup.

Subsurface defects may be detected using, for example, 2D or 3D near infrared imaging that penetrates into the volume of the teeth to reveal subsurface structures, such as the enamel and its thickness, the dentin location and its thickness, and dental caries. Tooth decay, wear, and/or damage may be determined based on changes to the patient's teeth over time, such as by detecting changes in the external shape of teeth across multiple external 3D and/or multiple subsurface 2D or 3D scans gathered over a period of time, such as once a year, for multiple years.

The 3D scan may reveal crowded or misaligned teeth that may lead to food traps or difficult to reach or clean locations on the teeth. Normal teeth are evenly spaced with mesial and distal edges of the teeth well-aligned along the upper and lower dental arches. Each tooth may be positioned to interact with its counterparts in the opposite jaw, facilitating efficient biting and chewing. The teeth are usually upright with their crowns and roots aligned in a way that supports healthy gum and bone structure. This alignment allows for a balanced distribution of biting forces, efficient mastication (chewing) of food, and helps in maintaining straightforward oral hygiene practices.

Crowded teeth, in contrast, may be characterized by a lack of space within the dental arches, leading to various alignment issues. The teeth may overlap each other in the mesial-distal or buccal-lingual directions with some teeth pushed and/or rotated in front of or behind their neighbors as they compete for space on the dental arch.

Some teeth might rotate partially around their axis, pointing in abnormal directions which disrupt the uniformity of the dental row. Such that, what would be their mesial-distal sides, may be arranged more buccal-lingually. Teeth in a crowded mouth may be tilted at various angles, either towards the tongue or the cheeks, rather than standing upright. This tilting can affect how the teeth meet when the jaws close, potentially leading to bite issues. Severely crowded conditions can force some teeth completely out of the normal alignment, leading them to jut out or recede significantly from the line of the other teeth.

These deviations from normal positioning complicate oral hygiene, as the irregular spacing and alignment create hard-to-clean areas, promoting plaque buildup and increasing the risk of dental decay and gum disease.

The system may detect teeth that one or more teeth or groups of teeth are crowded by determining their alignment or lack thereof based on the above factors or by comparison to properly arranged teeth.

A 2D or 3D scan may also reveal inflammation of the gingiva due to disease, surgery, dental implants, etc. Healthy gingiva, or gum tissue, typically presents as firm, pale pink, and tightly adhered to the underlying bone and teeth. It may have a smooth texture with a stippled appearance, often likened to the surface of an orange. The edges of healthy gingiva fit snugly around each tooth, forming a protective barrier that helps prevent the invasion of bacteria.

In contrast, gingival inflammation, medically known as gingivitis or periodontitis when more severe, manifests visibly and tactilely different traits. The inflamed gingiva often appears red or even purplish, swollen, and tends to bleed easily, particularly during brushing or flossing. The gingiva may also exhibit signs of formation of deep pockets between the teeth and gums. This condition results from the accumulation of bacterial plaque, which triggers an immune response leading to these characteristic changes.

When the gingiva is inflamed due to surgery, such as after a gingival graft, implant placement, or other periodontal procedures, it undergoes several changes. Initially, the surgical site may appear red, swollen, and tender to touch. This is a natural response as the body initiates the healing process. Unlike the uniform pink of healthy gingiva, the inflamed area might show varying shades of red or even purple, indicating increased blood flow and inflammatory activity.

The texture of the gingiva also changes significantly. Instead of being firm and tightly adhered, it may feel softer and more pliable. It may bleed easily, especially in the early days post-surgery, during brushing or when probed gently. As the healing progresses, granulation tissue may form, which is brighter red and softer, covering the wound to facilitate the regeneration of new tissue.

The 3D scanner may employ a structured light or other capture technology to capture the topography of the oral cavity. When scanning the oral cavity, including the gingiva, the scanner generates a precise 3D image if the gingiva. During the scanning process the 3D scanner may also capture color and textural data of the gingiva, which may be captured as 2D image data which may be mapped to the 3D model. The system may compare the captured 3D, color, and textural data to previous scans of the patient to determine if the patient's gingiva is inflamed. The system may analyze the captured 3D, color, and textural data to detect the presence red or purplish, swollen, gingiva and/or the formation of deep pockets between the teeth and gums to determine if the patient's gingiva is inflamed.

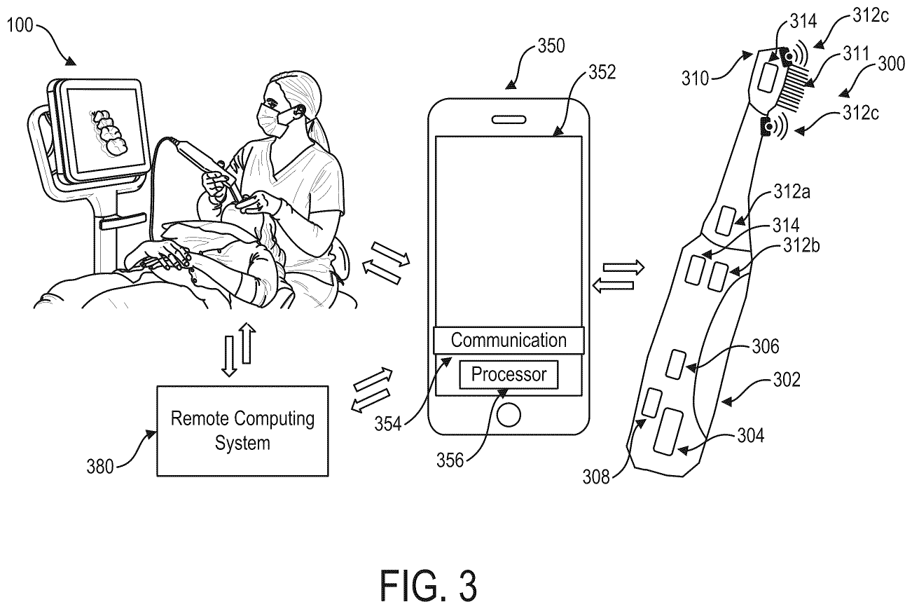

With reference to FIG. 3, a smart toothbrush 300 may be used to provide enhanced oral hygiene through technology integration. This smart toothbrush incorporates multiple subsystems, sensors, and software components to provide feedback and guidance to users, to aid in providing more effective and efficient brushing.

The handle 302 of the smart toothbrush includes an ergonomic design to facilitate easy gripping and maneuverability. The handle may house the main electronic components such as the controller 304 (which may be a microcontroller, processor, or other processing system), an energy storage device 308, such as a battery, and wireless communication module 306. The handle may be constructed from waterproof, durable materials capable of withstanding everyday use including use within the intraoral cavity and regular washing and rinsing.

The smart toothbrush 300 may include a brush head 310 that may be detachable from the handle 302 and includes bristles for effective cleaning and plaque removal. The head may include bristles 311 or one or of varying types, such as different bristles having different stiffness and length to cater to different dental needs. The brush head 310 may include sensors 312, such as sensors 312a which may include pressure sensors (such as a sensors that measures the strain on the toothbrush and uses the strain information to determine the force and/or pressure applied by the toothbrush to the teeth) and motion sensors, such as gyroscopes or accelerometers, to monitor brushing technique and force application.

The pressure sensor may be embedded in the neck or handle of the toothbrush. These sensor detects the amount of force the user applies when the brush head comes in contact with the teeth and gums. When the force exceeds a pre-set threshold, the sensor may activate a feedback or response mechanism.

The pressure sensor may be one or more types of sensors. In some embodiments, the pressure sensor may be a strain gauge. A strain gauge may include a conductive foil pattern that deforms when force is applied. This deformation changes the electrical resistance of the gauge, which can be measured and converted into a force reading. In some embodiments, the pressure sensor may be a piezoelectric sensor. Piezoelectric sensors use materials that generate an electric charge in response to applied mechanical stress. When embedded in a toothbrush, piezoelectric sensors can effectively measure the intensity of the brushing force based on the charge produced. In some embodiments, the pressure sensor may be a capacitive sensor. Capacitive sensors detect variations in electrical capacitance caused by the deformation of a capacitive plate due to applied pressure. In some embodiments, the pressure sensor may be a hall effect sensor. A hall effect sensor measures the magnetic field around them, which can be altered by the presence of a magnetic object. The smart toothbrush may include a small magnet that moves relative to the Hall effect sensor when force is applied to the brush head, changing the magnetic field and thus the sensor's output voltage which may be correlated to the pressure or force applied by the brush head to the teeth.

The sensors 312a may include one or more types of motion sensors. The motion sensors may include accelerometers and gyroscopes, these sensors track the orientation, direction, and speed of the brush as it moves within the mouth. The motion sensors may collect data which may be used to analyze the user's brushing pattern and coverage.

The toothbrush may also include one or more sensors 312c for sensing aspects of the environment around the toothbrush head, such as the intraoral environment. Sensors 312c may be 2D image sensors, such as cameras that capture two-dimensional images of the patient's intraoral cavity. In some embodiments, the sensors 312c may be ultrasound transceivers that emit and record reflected ultrasound data to generate distance data including ultrasound mapping data of the tooth surfaces relative to the toothbrush head.

By analyzing the sensor data, such as the acceleration data from the accelerometers and angular velocity data from the gyroscopes, the 2D image data, and/or the ultrasound data, the system can determine if the user is brushing in the correct locations, at the correct angles, and using the recommended motions based on correlation of the gathered tooth brushing data with the 3D intraoral scan data, as discussed herein. Determining position, orientation, and motions helps confirm whether or not the surfaces of the teeth and gums are properly cleaned. The data collected by the sensors can be used to provide real-time feedback to the user via a connected app. For example, if the user is brushing too hard, not brushing long enough, or missing certain areas, the app can alert the user and suggest improvements. The data collected by the sensors can be used to provide real-time feedback to the user via a connected app on a smart device, such as a smartphone 350. For example, if the user is brushing too hard, not brushing long enough, or missing certain areas, the system can alert the user and suggest improvements.

Over time, the system can map the user's brushing patterns and identify consistent missed spots or suboptimal techniques. This information may be used by the system, the user, and dental professionals to provide feedback and improve oral hygiene routines.

The handle 302 may also include sensors 312, such as sensors 312b that may be any of the sensors described herein, such as those described with respect to the brush head sensors 312a. The sensors 312b may be used to aid in determining the position, orientation, and/or motion of the handle 302 and to aid in determining how the user is using the toothbrush and how effective the user is brushing their teeth.

The toothbrush 300 may include a motor 314 for moving the brush head 310 during brushing. The motors may be located in the handle 302 or in the head assembly 310. The motors may be rotary motors, sonic or ultrasonic vibratory motors, or other types of motors. Rotary motors, such as DC or brushless motors may connect to a gear and cam system that translates the motor's rotational movement into the brush head's and/or its bristles into an oscillating action. The motor may cause the bristles to rotate in one direction and then the other, such as by making quarter-turns (90 degree rotations) back and forth to dislodge plaque and stimulate the gums effectively.

A vibratory motor may utilize a piezoelectric effect of a piezoelectric crystal such that when an electric current is applied to the motor, it vibrates at an ultrasonic frequency (such as above 20,000 Hz). Other vibratory motors may use electromagnets and permanent magnets to cause vibrations in the toothbrush head. The vibrations are transferred to the brush head, which then transmits these rapid movements to the bristles.

The toothbrush 300 may also include a communication system 306. The communication system may communicate wirelessly, such as through Bluetooth or Wi-Fi, to interface with mobile devices or a dedicated display unit 350. This subsystem enables data transfer between the toothbrush sensors and/or controller 304 and the mobile device 350 for analysis and feedback by the system.

The toothbrush 300 may also include an energy system 308 for storing and providing energy to the various elements of the toothbrush. For example, the toothbrush may be powered by a battery, such as a rechargeable lithium-ion battery, or a capacitor. The handle 302 may contain a charging interface, such as at it that connects to a wireless charging dock or a USB charging cable for charging the energy source.

The system may also include a smart computing device 350, which may be a smartphone. The smart computing device may include a display 352 which may display a user interface including feedback, dental information, brushing information, and other data, such as described herein.

The computing device 350 may also include a communication system 354. The communication system may communicate wirelessly, such as through Bluetooth or Wi-Fi, to interface with the smart toothbrush 300. This subsystem enables data transfer to and from the controller 354 of the mobile device 350 and the toothbrush 300.

The computing device 350 may also include a controller 356 having a processor and memory as described herein.

The computing device 350 may execute instructions that cause the system to perform any of the tasks or functions described herein. For example, the computing device may include instructions for an application, such as a mobile application that may receive or analyze data received from the toothbrush. The application may provide a user-friendly interface displaying detailed analytics on brushing habits, such as duration, frequency, pressure, and coverage. The application may analyze the incoming data to generate personalized feedback and brushing recommendations. It can track progress over time, suggest changes or improvements in brushing techniques, and remind users of their suggested brushing plan as provided by the dentist such as generated by a treatment planning system.

In some embodiments, the system further includes a remote computing system 380. The remote computing system 380 may be in communication with the system 100, the mobile device 350, and the smart toothbrush 300 via one or more wired or wireless communication networks. The remote computing system 380 may receive data from one or more of the system 100, the mobile device 350, and the smart toothbrush 300, including brushing data, user interaction data, compliance data, oral health data, guidance data, and or other data described herein.

In some embodiments, the remote computing system 380 is configured to process, analyze, and store the received data and to perform one or more functions described herein that may alternatively or additionally be performed by the system 100, the mobile device 350, or the smart toothbrush 300. For example, the remote computing system 380 may analyze brushing patterns, monitor compliance with brushing guidance, generate or update customized treatment plans, generate alerts or feedback, implement gamification features, or generate reports for patients or dental professionals.

In some embodiments, the remote computing system 380 supports centralized data aggregation and analysis across multiple brushing sessions or multiple users. The remote computing system 380 may also facilitate communication between the patient-facing components and dental practice systems, including transmitting compliance information, treatment plan updates, or alerts to dental professionals.

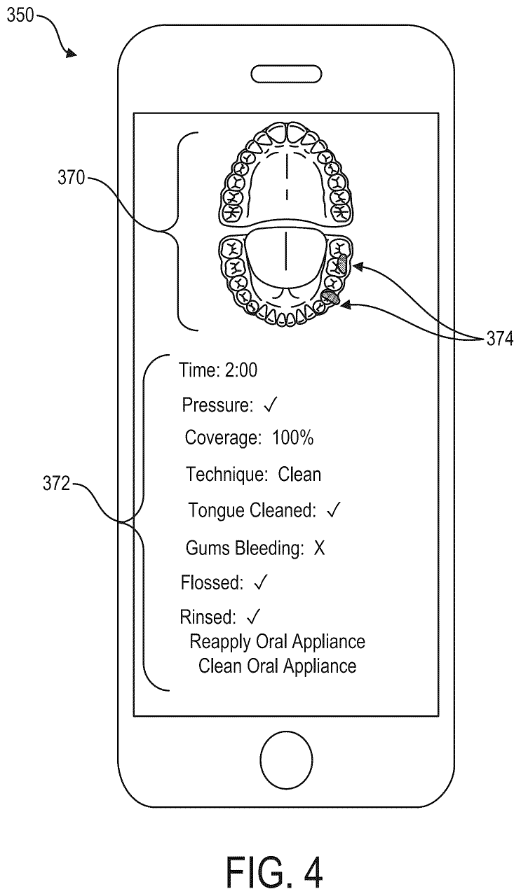

FIG. 4 depicts an embodiment of a smart device 350 displaying a smart toothbrush user interface. The smart device visually and interactively assists users in monitoring and improving their brushing, displaying problem areas as determined by intraoral scanning, and other information.

The smart device may display detailed occlusal or other views 370 of 2D or 3D models of the upper and lower dental arches. Areas of concern or other areas or locations 374 on the dentition, such locations as potential cavities, plaque accumulation, crowded teeth, gingival swelling, or signs of gum disease, are highlighted or otherwise called out on the model and may be indicated by colors, flashing signs, or pop-up annotations that provide more information when tapped or otherwise interacted with by a user.

The views 370 may also include one or more heat maps, such as a pressure heatmap and a brushing coverage heat map. The pressure heat map may overlay the 2D or 3D dental views to show where excessive or too little pressure was or is applied during brushing. The heat map may be generated in real time to provide feedback to the user. The heat map may include a color gradient over the 2D or 3D view that ranges from, for example, deep red (to indicate too much pressure) to deep blue (to indicate too little pressure), with a green color, for example, indicating a pressure within a range of acceptable brushing pressure.

A coverage heat map may overlay on the 2D or 3D view that uses color coding to show how well each tooth surface was brushed. The heat map may be generated in real time to provide feedback to the user. For example, green indicates good coverage, orange may signify partial coverage, and red or no color may indicate areas that were missed completely or still need to be brushed. Touch interaction on the screen with the model may allow users to rotate the 2D or 3D dental models, zoom in for detailed views, and tap areas of interest, such as areas of concern, poor brushing, tooth defects, etc. for more information, such as brushing guidance or directions, etc.

The smart device 350 may also display analytical data and health tracking data 372. For example, the device may display analytical data collected during toothbrushing.

A session timer may display the duration of the latest brushing session. The timer may also provide guidance regarding recommended times, such as a count up or count down timer representing the time left or total time spent brushing. The timer may also include a historical trend line for past sessions.

An indication of brush pressure may be included that includes a statistical breakdown of how often the suggested brushing pressure or pressure range was maintained, including a percentage score, a check mark, an X, or other indication that the pressure was within a suggested range or not within a suggested range and for how long. The pressure feedback may be provided in real time, such as based on sensor data from the toothbrush during brushing.

An indication of coverage effectiveness may be provided. The coverage effectiveness may summarize the completeness of a brushing session in real time during a brushing session or after a session is complete.

An indication of the brush settings or technique may also be displayed. The indication may include details the specific modes and settings used during a brushing session, such as gum care or whitening modes, and may also indicate any adjustments or deviations from suggested settings or techniques that might enhance brushing effectiveness.

The device may also collect information from a user and/or the toothbrush regarding whether or not the tongue was cleaned, whether the gums were bleeding and where, whether or not the teeth were flossed, and whether or not the mouth was rinsed with mouthwash or other fluids.

For patients undergoing orthodontic treatment with aligners or other oral appliances, the display may also include a reminder to reapply the oral appliance or appliances after brushing.

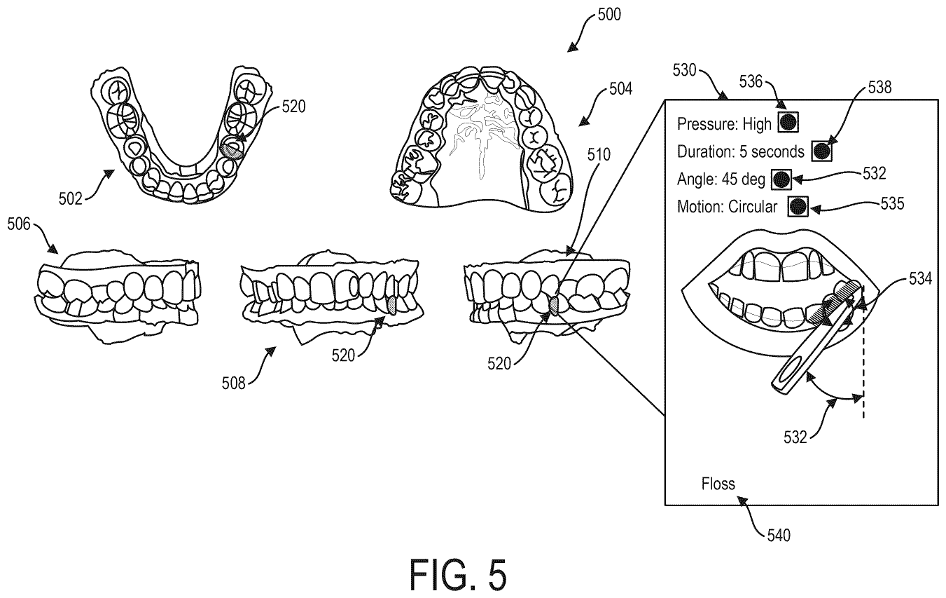

FIG. 5 depicts detailed views 500 of 2D or 3D models of the upper and lower dental arches. The views 500 may include an occlusal view 502 of the lower arch, an occlusal view 504 of the upper arch, a right buccal view 506 of the upper and lower arches in occlusion, a midline view 508 of the upper and lower arches in occlusion, and a right buccal view 510 of the upper and lower arches in occlusion. An area or location 520 where instructions may be provided for a deviation from normal brushing techniques. For example, such locations may include potential cavities, plaque accumulation, crowded teeth, gingival swelling, or signs of gum disease, may be highlighted or otherwise called out on the model and may be indicated by colors, flashing signs, or pop-up annotations that provide more information when tapped or otherwise interacted with by a user. These may be areas or locations where intraoral scanning and imaging may have revealed a dental issue that may benefit from different brushing techniques.

Interacting with the area or location 520 may cause the system to provide a display 530 of the recommended brushing technique. The display may include a depiction of the angle 532 at which the brush is to be held, a motion 534 that the user should make with the toothbrush while brushing, and indication 536 of how much pressure to apply, and a duration 538 that the user should brush at a particular location. The system may also provide feedback during brushing as to whether or not the user is brushing according to the directions. For example, the feedback may include a countdown or count up timer while the user is brushing their teeth at the particular location and an indication that the directed bushing time has been achieved.

During brushing, the depiction 536 may also include an indication or feedback as to whether or not the pressure applied to the brush head is within the directed range, such as a colored indication. The indication may also include information as to whether the pressure is too high or too low or whether to reduce or increase pressure, such as by using an up arrow to indicate too much pressure or to increase pressure or a down arrow to indicate too little pressure or to increase pressures. The feedback may include direction to increase, decrease, or maintain pressure.

During brushing, the depiction 532 may also include an indication or feedback as to whether or not the toothbrush is being held at the appropriate angle, such as a colored indication. The indication may also include information as to whether the angle is too high or too low or whether to reduce or increase the angle, such as by using an up arrow to indicate in which direction to rotate the brush to achieve the correct angle or a display of the current angle. The feedback may include direction to increase, decrease, or maintain the brush angle.

During brushing, the depiction 534 may also include an indication or feedback as to whether or not the toothbrush is being moved in the directed manner, such as a colored indication. The indication may provide feedback as to the current brushing technique being detected and feedback as to how to correct the movement.

The feedback or directions may also include directions 540 on additional oral care to be used at the particular location, such as use of floss, a water pick, or other care and an interface 540 for the user to interact with to confirm they directions were followed.

The system may provide the feedback based on sensor data received while brushing. The system may determine the location of the toothbrush within the mouth and which teeth are being brushed based on the sensor data. The system may also determine the pressure applied by the brush head to the teeth, the angle of the toothbrush, and other information descried herein based on the sensor data. For example, the duration timer may only update when the toothbrush is detected to be at the directed location and/or at the correct angle and pressure and with the correct motion.

For example, when the system determines that there is plaque buildup at the bicuspid, the system may provide an indication 520 at the location of the bicuspid. Interacting with the indication 520 causes the system to display graphics 530 with instructions and depictions for addressing plaque removal. For example, for improved plaque removal that system may indicate that the patient should use relatively high pressure, for five seconds on each of the buccal, lingual, and occlusal surfaces while holding the toothbrush at 45 degrees while on the buccal side. Angling the head of the toothbrush at about a 45-degree angle towards the gums and also relative to the vertical direction. This positioning allows the bristles to reach the gingival sulcus, the area beneath the gum line where plaque accumulates. The angled bristles can gently sweep away the plaque when the circular motions are applied.

For other detected issues the system may provide different depictions and instructions. For example, for inflammation due to recent surgery, such as a dental implant, instead of the standard circular or vertical motions, the system may recommend a gentle tapping motion with the brush. This minimizes the movement against the inflamed gums, reducing the likelihood of causing irritation while still helping to remove plaque from the tooth surfaces adjacent to the surgical site while avoiding aggressive back-and-forth or circular brushing motions as they can further irritate and damage sensitive gum tissue. The system may provide guidance to hold the toothbrush bristles parallel to the line of the gums rather than the typical 45-degree angle. This approach may help prevent the bristles from directly contacting the inflamed surgical site while still allowing some cleaning of the tooth surfaces. The system may provide guidance to use very light pressure to avoid aggravating the inflamed gums. The system may also automatically set the smart toothbrush to a low or its lowest setting and possibly consider switching to a brush head designed for sensitive gums. The system may also provide guidance regarding the time spent brushing at the surgical such, such as less than 5 seconds and to avoid flossing and alcohol-based mouth washing. The system may also provide guidance 540 to use a saltwater rinse.

For crowded teeth the system may provide guidance 534 to use vertical strokes in an occlusal-gingival direction between the gums and the occlusal portion of the teeth to help the bristles reach between the crowded areas better than horizontal strokes. The system may provide guidance 534 to provide circular motions in addition to the vertical motions in areas with crowded teeth. The time brushing a crowded tooth or teeth may be increased, such as by providing guidance 538 to increase the brushing time to 10 seconds for a crowded tooth. Guidance 540 may also be provided to use interdental brushes, floss, or water flossers to remove plaque and debris that a toothbrush might miss due to the tight spaces between crowded teeth.

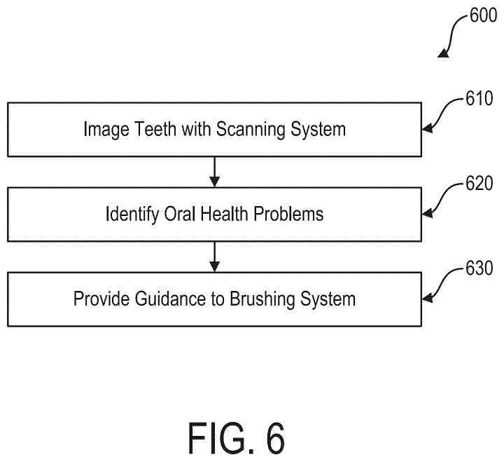

FIG. 6 depicts a method 600 for using intraoral imaging to determine oral health problems and provide brushing guidance. The method 600 may include imaging teeth at block 610, identifying dental health problems at block 620, and providing brushing guidance at block 630.

At block 610, the intraoral cavity of the patient may be imaged to generate image data of the patient's oral cavity. Imaging may include 2D and/or 3D imaging of the patient's teeth, gums, and subsurface tissue of the teeth and gums. During the imaging process, as the scanner is manually maneuvered throughout the mouth, the scanner captures a multitude of images from various angles.

As the scanner captures images, point cloud data is generated from each of the images. Each point within these clouds represents a specific location on the surface of the dentition, encoded in three-dimensional coordinates. These points are derived from the reflections and distortions of the scanner's light as it interacts with the dental surfaces, which the scanner's sensors and cameras capture and convert into digital data sets of point clouds.

The point clouds are stitched together. The stitching process involves the alignment and integration of multiple point clouds to assemble a three-dimensional model of the oral cavity including, for example 3D models of the upper and lower dentition. The process includes detecting similar 3D structures in overlapping portions of the point clouds and aligning the point clouds with each other based on these overlapping areas.

The 3D digital model, derived from the intraoral scanner's data, can be used in various dental applications, including for example, orthodontic, restorative, and other dental treatment planning processes, the creation of orthodontic aligners, dental crowns, and bridges, the assessment of oral health, and other processes.

During the scanning process, 2D images of the patient's oral cavity, such as the dentition, including the teeth, and the gums may be captured. The 2D images may include 2D color surface images, such as those captured with white light or other visible light illumination, and 2D subsurface images, such as those captured with near-IR illumination.

At block 620, the image data of the patient's oral cavity may be analyzed to identify locations of oral health problems. The system may detect dental defects, such as tooth decay, plaque buildup and/or dental caries (cavities), such as by using a plaque or caries stain before scanning the teeth or other dental and oral health issues. For example, the 2D images captured with the intraoral scanner of stained teeth. For example, a caries dye or disclosing solutions may have been applied to the teeth before or during the imaging process. The areas of decay absorb the stain and appear differently colored compared to the healthy enamel. For instance, a common color for these stains is red or blue, which provides a sharp contrast to the natural color of the tooth, making the carious lesions more visible. The 3D scan may capture 3D and color data for the teeth with the dyed or stained locations on the teeth indicating potential locations of caries. The system may identify locations of caries based on the location of the dye on the teeth.

Similar to detection of caries, stains may also be used to detect dental plague. A dye or stain that that selectively adheres to plaque, with little to no adherence to the clean surfaces of teeth may have been applied to the teeth before or during imaging. The dye binds to the plaque deposits, staining them a vivid color (commonly pink, red, or blue) that contrasts sharply with the natural color of teeth, making the plaque clearly visible. The areas of with plaque buildup absorb the stain and appear differently colored compared to the cleaned portions of the teeth which provides a sharp contrast to the natural color of the tooth, making the plaque more visible. The imaging may capture color data for the teeth with the dyed or stained locations on the teeth indicating potential locations of dental plaque buildup. The system may identify locations of caries based on the location of the dye on the teeth.

Subsurface defects may be detected using, for example near infrared imaging that penetrates into the volume of the teeth to reveal subsurface structures, such as the enamel and its thickness, the dentin location and its thickness, and dental caries. Tooth decay, wear, and/or damage may be determined based on changes to the patient's teeth over time, such as by detecting changes in the external shape of teeth across multiple 3D scans gathered over a period of time, such as once a year, for multiple years.

The 3D scan may reveal crowded or misaligned teeth that may lead to food traps or difficult to reach or clean locations on the teeth. Normal teeth are evenly spaced with mesial and distal edges of the teeth well-aligned along the upper and lower dental arches. Each tooth may be positioned to interact with its counterparts in the opposite jaw, facilitating efficient biting and chewing. The teeth are usually upright with their crowns and roots aligned in a way that supports healthy gum and bone structure. This alignment allows for a balanced distribution of biting forces, efficient mastication of food, and helps in maintaining straightforward oral hygiene practices.

Crowded teeth, in contrast, may be characterized by a lack of space within the dental arches, leading to various alignment issues. The teeth overlap may overlap each other in the mesial-distal or buccal-lingual directions with some teeth pushed in front of or behind their neighbors as they compete for space on the dental arch.

Some teeth might rotate partially around their axis, pointing in abnormal directions which disrupt the uniformity of the dental row. Such as what would be their mesial-distal sides being arranged more buccal-lingually. Teeth in a crowded mouth may be tilted at various angles, either towards the tongue or the cheeks, rather than standing upright. This tilting can affect how the teeth meet when the jaws close, potentially leading to bite issues. Severely crowded conditions can force some teeth completely out of the normal alignment, leading them to jut out or recede significantly from the line of the other teeth.

These deviations from normal positioning complicate oral hygiene, as the irregular spacing and alignment create hard-to-clean areas, promoting plaque buildup and increasing the risk of dental decay and gum disease.

The system may detect teeth that are crowded by determining their alignment or lack thereof based on the above factors or by comparison to properly arranged teeth.

The 3D scan may also reveal inflammation of the gingiva due to disease, surgery, dental implants, etc. Healthy gingiva, or gum tissue, typically presents as firm, pale pink, and tightly adhered to the underlying bone and teeth. Healthy gingiva may have a smooth texture with a stippled appearance, often likened to the surface of an orange. The edges of healthy gingiva fit snugly around each tooth, forming a protective barrier that helps prevent the invasion of bacteria.

In contrast, inflamed gingiva often appears red or even purplish, swollen, and tends to bleed easily, particularly during brushing or flossing. The gingiva may also exhibit signs of formation of deep pockets between the teeth and gums. This condition results from the accumulation of bacterial plaque, which triggers an immune response leading to these characteristic changes.

When the gingiva is inflamed due to surgery, such as after a gingival graft, implant placement, or other periodontal procedures, it undergoes several changes. Initially, the surgical site may appear red, swollen, and tender to touch. This is a natural response as the body initiates the healing process. Unlike the uniform pink of healthy gingiva, the inflamed area might show varying shades of red or even purple, indicating increased blood flow and inflammatory activity.

The texture of the gingiva also changes significantly; instead of being firm and tightly adhered, it may feel softer and more pliable. It may bleed easily, especially in the early days post-surgery, during brushing or when probed gently. As the healing progresses, granulation tissue may form, which is brighter red and softer, covering the wound to facilitate the regeneration of new tissue.

The 3D scanner may employ a structured light or other capture technology to capture the topography of the oral cavity. When scanning the oral cavity, including the gingiva, the scanner generates a precise 3D image if the gingiva. During the scanning process the 3D scanner may also capture color and textural data of the gingiva. The system may compare the captured 3D, color, and textural data to previous scans of the patient to determine if the patient's gingiva is inflamed. The system may analyze the captured 3D, color, and textural data to detect the presence red or purplish, swollen, gingiva and/or the formation of deep pockets between the teeth and gums to determine if the patient's gingiva is inflamed.

At block 630, guidance may be provided for toothbrushing for each of the identified locations of oral health problems. The guidance may be provided to the smart device 350 or directly to the toothbrush. The guidance may include aspects of how to brush teeth based on the detected defect or oral health issue. The guidance may include a depiction of the angle at which the brush is to be held, a motion that the user should make with the toothbrush while brushing, and indication of how much pressure to apply, and a duration that the user should brush at a particular location.

For example, when the system determines that there is plaque buildup at the bicuspid, the system may provide guidance for how to brush teeth at the location of the bicuspid. The guidance may include graphics with instructions and depictions for addressing plaque removal. For example, for improved plaque removal that system may provide guidance that the patient should use relatively high pressure, for five seconds on each of the buccal, lingual, and occlusal surfaces while holding the toothbrush at 45 degrees while on the buccal side. Angling the head of the toothbrush at about a 45-degree angle towards the gums and also relative to the vertical direction. This positioning allows the bristles to reach the gingival sulcus, the area beneath the gum line where plaque accumulates. The angled bristles can gently sweep away the plaque when the circular motions are applied.

For other detected issues the system may provide different depictions and instructions. For example, for inflammation due to recent surgery, such as a dental implant, instead of the standard circular or vertical motions, the system may recommend a gentle tapping motion with the brush. This minimizes the movement against the inflamed gums, reducing the potential to cause irritation while still helping to remove plaque from the tooth surfaces adjacent to the surgical site while avoiding aggressive back-and-forth or circular brushing motions as they can further irritate and damage sensitive gum tissue. The system may provide guidance to hold the toothbrush bristles parallel to the line of the gums rather than the typical 45-degree angle. This approach may help prevent the bristles from directly contacting the inflamed surgical site while still allowing some cleaning of the tooth surfaces. The system may provide guidance to use very light pressure to avoid aggravating the inflamed gums. The system may also automatically set the smart toothbrush to a low or its lowest setting and possibly consider switching to a brush head designed for sensitive gums. The system may also provide guidance regarding the time spent brushing at the surgical such, such as less than 5 seconds and to avoid flossing and alcohol-based mouth washing. The system may also provide guidance to use a saltwater rinse.

For crowded teeth the system may provide guidance to use vertical strokes in an occlusal-gingival direction between the gums and the occlusal portion of the teeth to help the bristles reach between the crowded areas better than horizontal strokes. The system may provide guidance to provide circular motions in addition to the vertical motions in areas with crowded teeth. The time brushing a crowded tooth or teeth may be increased, such as by providing guidance to increase the brushing time to 10 seconds for a crowded tooth. Guidance may also be provided to use interdental brushes, floss, or water flossers to remove plaque and debris that a toothbrush might miss due to the tight spaces between crowded teeth.

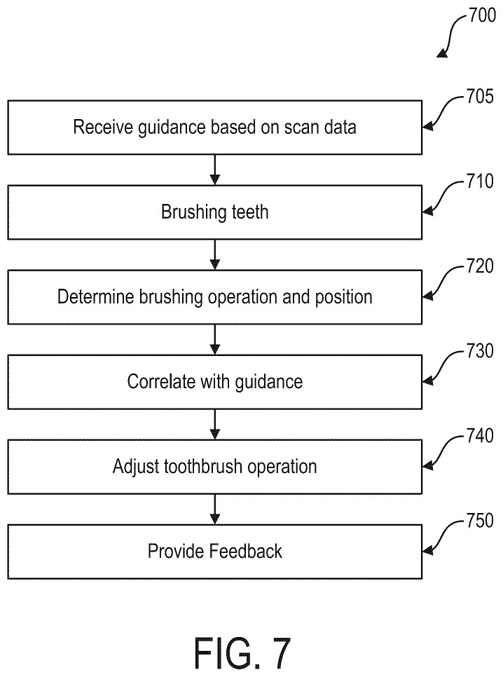

FIG. 7 depicts a method 700 for brushing teeth and providing tooth brushing feedback while a patient brushes their teeth. The method may include receiving guidance generated based on the intraoral scanning system 705, operating a smart toothbrush to brush teeth at block 710, determining brushing operation at block 720, correlating the brushing operation with guidance at block 730, adjusting toothbrush operation at block 740, and providing feedback or updated guidance at block 750.

At block 705, the system may receive guidance information and the 3D model. For example, the smart device 350 or the toothbrush may receive guidance information and the 3D model generated based on the data collected by the scanning system 100.

At block 710, a smart toothbrush may begin brushing a patient's teeth. A patient may move the smart toothbrush around within the oral cavity brushing buccal, lingual, and occlusal surfaces of the teeth and gums. In some embodiments, brushing may begin automatically when the system detects that the tooth brush is in the correct location and/or orientation and/or brush pressure.

During the brushing operation, at block 720, the method may include determining the qualities of the tooth brushing operation using sensors and other features and elements of a smart toothbrush. The system may determine the position of the toothbrush relative to the detention, the force or pressure applied by the toothbrush to the teeth and gums, the angle of the toothbrush, the time spent brushing at each location of the teeth and gums, the motion of the toothbrush at each location, and the operational mode of the toothbrush at each location.

Toothbrush position and orientation may be determined based on the sensor data, such as using gyroscopes and accelerometers to track the position and orientation. In some embodiments, the tracking may be from a known position and orientation. A known position and orientation may be determined in many ways, such as using sensor data, including image or ultrasound data correlated to the 3D model generated using intraoral scan data.

In some embodiments, the 3D model of the intraoral cavity is provided. This model may be obtained, for example, from dental imaging such as CT (Computed Tomography) scans, MRI (Magnetic Resonance Imaging), and/or intraoral scans acquired using optical, laser, or structured-light scanners, such as the intraoral scanning system discussed herein. The model may be stored in a as a mesh, volumetric voxel representation, or a surface-based polygonal model. The model may include known geometrical features, landmarks, textures, or annotated fiducial markers.

A 2D image may be obtained from an image sensor integrated into or otherwise coupled to the toothbrush, such as sensors 312c. This image may be a digital photograph or video frame depicting a portion of the patient's intraoral cavity, such as the surface of a tooth, gingiva, cheek, or another portion of the oral cavity, or another visible anatomical feature. The image sensor may be a CCD, CMOS, or equivalent camera device configured to capture still images or a continuous video feed at known resolution and known camera parameters.

To facilitate alignment of the sensor data with the 3D model, the system may identify one or more features in the sensor data, such as in 2D image or ultrasound data, that correspond to known anatomical structures, such as surface features represented in the 3D model. These features may include landmarks, such as prominent anatomical structures, such as edges, corners, ridges, or well-defined points on the anatomy (e.g., a particular tooth prominence, stain, or a unique tooth cusp). The features may also be texture or patterns on or in the anatomy such as surface patterns, coloration gradients, or identifiable textures visible in the 2D image that can be matched to texture maps associated with the 3D model.

The feature identification may be accomplished by employing image processing algorithms, such as edge detection, feature extraction (e.g., using SIFT, SURF, or ORB descriptors), template matching, or machine learning-based classification, to determine the presence and coordinates features, which may be key points within the 2D image.

Each identified feature in the 2D image may then be associated with a corresponding location on the 3D model. The 3D model may be defined in a known coordinate system, such that identifying a matching anatomical feature in the model yields a set of correspondences between 2D image coordinates and 3D anatomical coordinates. This step may rely on a pre-computed database of anatomical landmarks or, in the absence of distinct landmarks, a best-fit approximation based on comparing the captured 2D data with a library of rendered 2D projections of the 3D model.

Using the established correspondences, an initial pose (e.g., the position and orientation) of the image sensor relative to the 3D anatomical coordinate system may be computed. This computation may include solving a camera pose estimation problem to determine the camera's rotation and translation matrices from a set of 2D-to-3D matched points to generate, for example, a transformation matrix that, when applied to the 3D model, simulates how the model would be seen from the camera's viewpoint.

After the initial pose is estimated, the method may refine and optimize the alignment to minimize positioning errors. For example, the system may iteratively refine correspondences, such as by identifying additional features in the 2D image, reject outlier correspondences, and use a more comprehensive set of matched points to improve accuracy. In some embodiments, the method may include applying error minimization techniques, such as employing non-linear optimization algorithms, such as the Levenberg-Marquardt or Gauss-Newton algorithms, to minimize a reprojection error metric. Reprojection error measures how closely the 3D model's projected features align with the actual 2D image features. In some embodiments, the method may incorporate image-based rendering comparisons wherein the system may render synthetic 2D images of the 3D model from the currently estimated pose and compare these synthetic images to the acquired 2D image. By adjusting the pose to reduce differences in contour, intensity gradients, or texture alignment, the registration can be further refined.

In some embodiments, alignment may be validated by checking consistency across multiple frames of image data, such as 2D image data, or by comparing known distances and anatomical relationships in the model against what can be inferred from the 2D image. The registration may be considered successful if the distances and relationships are below a predefined threshold.

Once the 2D image is aligned to the 3D model, the pose of the toothbrush imaging sensor is known within the anatomical coordinate system. This pose information can then be used to determine the location and orientation of the toothbrush relative to the patient's intraoral cavity based on the image sensors known position and orientation with respect to the toothbrush, which may be used to provide guidance and/or control the toothbrush operation in real-time during the toothbrushing process. In some embodiments, this pose data may be recorded for future reference and/or use as discussed herein.

The sensor may continuously or repeatedly capture new images during the toothbrushing process. The method for determining location and orientation of the toothbrush may be repeated or updated in real-time. The system can perform continuous alignment adjustments as the brush moves, thereby providing real-time guidance to the operator and additional location data for later use.

At block 730, the method may include correlating the toothbrush location with guidance, such as the guidance generated using the method of FIG. 6. Based on the sensor data the method may determine the location of the toothbrush and correlate that location with the location of guidance, such as the guidance generated using the method of FIG. 6. The method may include comparing the force or pressure applied by the toothbrush to the teeth and gums, the angle of the toothbrush, the time spent brushing at a location of the teeth and gums, the motion of the toothbrush at the location, and the operational mode of the toothbrush at the location. The system may compare these with the guidance and determine if the toothbrush operation should be adjusted and/or whether to provide feedback or updated guidance to the user during brushing.

For example, at block 740, the method may include adjusting the operation of the toothbrush. For example, when the method detects that the toothbrush is near a location of a surgical site, the toothbrush may change to a low setting, such as a slower movement setting or lower power setting, then when the method determines that the brush is no longer at the location of a surgical site, the toothbrush may change to a higher setting, such as a faster movement setting or a higher power setting. Any of the operational settings described herein may be modified during brushing, such as by starting, stopping, increasing, or decreasing operation of the toothbrush.

For example, at block 750, the method may include providing feedback during the operation of the toothbrush. The data collected by the sensors can be used to provide real-time feedback to the user. For example, if the user is brushing too hard, not brushing long enough, or missing certain areas, the method can alert the user and suggest improvements. The data collected by the sensors can be used to provide real-time feedback to the user via a connected app on a smart device, such as a smartphone. For example, if the user is brushing too hard, not brushing long enough, or missing certain areas, the system can alert the user and suggest improvements.

The system may also provide feedback during brushing as to whether or not the user is brushing according to the directions. For example, the feedback may include a countdown or count up timer while the user is brushing their teeth at the particular location and an indication that the directed bushing time have been achieved.

The feedback may also include an indication as to whether or not the pressure applied to the brush head is within the directed range, such as a colored indication. The indication may also include information as to whether the pressure is too high or too low or whether to reduce or increase pressure, such as by using an up arrow to indicate too much pressure or to increase pressure or a down arrow to indicate too little pressure or to increase pressures. The feedback may include direction to increase, decrease, or maintain pressure.

The feedback may also include an indication as to whether or not the toothbrush is being held at the appropriate angle, such as a colored indication. The indication may also include information as to whether the angle is too high or too low or whether to reduce or increase the angle, such as by using an up arrow to indicate in which direction to rotate the brush to achieve the correct angle or a display of the current angle. The feedback may include direction to increase, decrease, or maintain the brush angle.

The feedback may also include an indication as to whether or not the toothbrush is being moved in the directed manner, such as a colored indication. The indication may provide feedback as to the current brushing technique being detected and feedback as to how to correct the movement.

The feedback or directions may also include directions on additional oral care to be used at the particular location, such as use of floss, a water pick, or other care and an interface for the user to interact with to confirm they directions were followed.

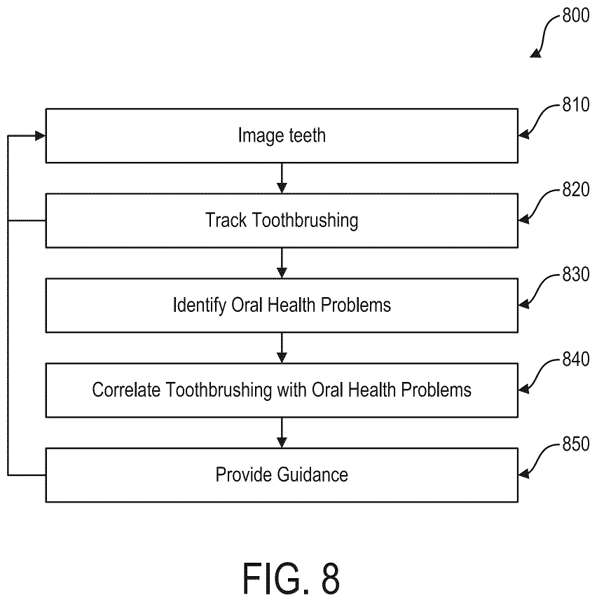

FIG. 8 depicts a method 800 for tracking toothbrushing and oral health and providing guidance based thereon. The method 800 may include imaging teeth at block 810, tracking toothbrushing at block 820, identifying health problems at block 830, correlating toothbrushing with oral health problems at block 840, and providing guidance and treatment at block 850.

At block 810 the intraoral cavity of the patient may be imaged to generate image data of the patient's oral cavity. Imaging may include 2D and/or 3D imaging of the patient's teeth, gums, and subsurface tissue of the teeth and gums. During the imaging process, as the scanner is manually maneuvered throughout the mouth, the scanner captures a multitude of images from various angles.

As the scanner captures point cloud data is generated from each of the images. Each point within these clouds represents a specific location on the surface of the dentition, encoded in three-dimensional coordinates. These points are derived from the reflections and distortions of the scanner's light as it interacts with the dental surfaces, which the scanner's sensors and cameras capture and convert into digital data sets of point clouds.

The point clouds are stitched together. The stitching process involves the alignment and integration of multiple point clouds to assemble a three-dimensional model of the oral cavity including, for example 3D models of the upper and lower dentition. The process includes detecting similar 3D structures in overlapping portions of the point clouds and aligning the point clouds with each other based on these overlapping areas.

The 3D digital model, derived from the intraoral scanner's data, can be used in various dental applications, including for example, orthodontic, restorative, and other dental treatment planning processes, the creation of orthodontic aligners, dental crowns, and bridges, the assessment of oral health, and other processes.

During the scanning process, 2D images of the patient's oral cavity, such as the dentition, including the teeth, and the gums may be captured. The 2D images may include 2D color surface images, such as those captured with white light or other visible light illumination, and 2D subsurface images, such as those captured with near-IR illumination.

At block 820, tooth brushing may be tracked. During each toothbrushing session, the method may include determining the qualities of the toothbrushing operation using sensors and other features and elements of a smart toothbrush. The system may determine the position of the toothbrush relative to the detention, such as described with respect to block 720 of method 700, the force or pressure applied by the toothbrush to the teeth and gums, the angle of the toothbrush, the time spent brushing at each location of the teeth and gums, the motion of the toothbrush at each location, and the operational mode of the toothbrush at each location.

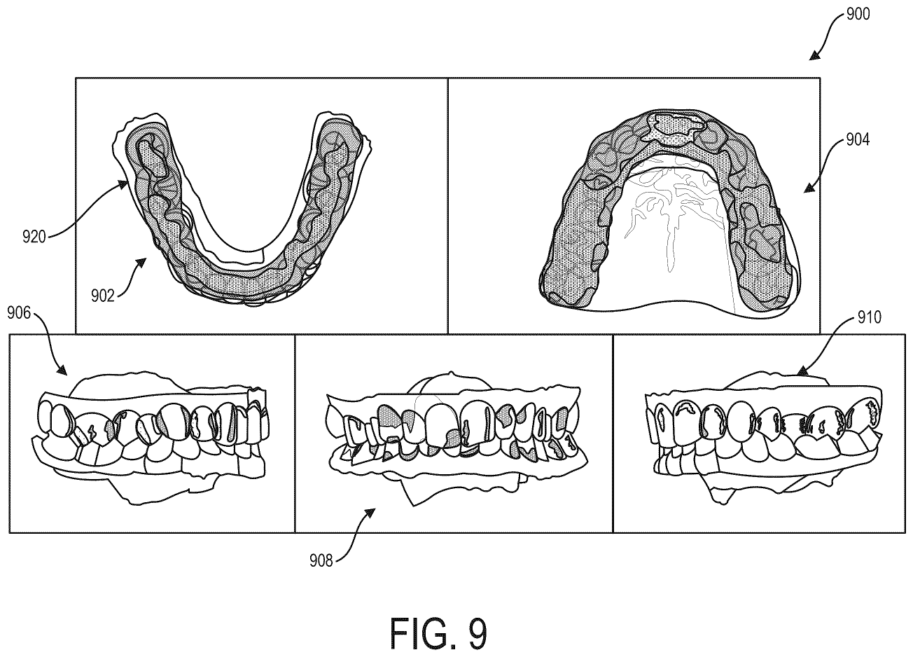

FIG. 9 depicts an example of toothbrushing data tracked overtime. FIG. 9 depicts detailed views 900 of 2D or 3D models of the upper and lower dental arches with a depiction of toothbrushing data 920 tracked over time. The views 900 may include an occlusal view 902 of the lower arch, an occlusal view 904 of the upper arch, a right buccal view 906 of the upper and lower arches in occlusion, a midline view 908 of the upper and lower arches in occlusion, and a right buccal view 910 of the upper and lower arches in occlusion.

The toothbrushing data may take the form of a heat map. The heat map may depict aspects of the patient's tooth brushing history of a period of time, such as single brushing session or multiple brushing sessions over a day, week, month, year, etc. The color of the heat map may correspond to the overall adequacy of the brushing over time taking into account the time spent brushing, the pressure applied, the technique used, and toothbrush settings, and the other aspects of toothbrushing discussed herein. In some embodiments, the data displayed or the heat map may be based on less than all of the aspects of toothbrushing, such as one or more of the pressure applied to each location, the time spent at each location, whether or not the proper technique or toothbrush setting was applied at each location, and the other aspects of toothbrushing discussed herein.

The tracking of toothbrushing over time may also include tracking the toothbrushing efficiency based on an efficiency score that is the area of teeth covered divided by brushing time. This may measure how well a patient covers the oral area withing an optimal time. A higher score suggests better brushing efficiency.

The tracking of toothbrushing over time may also include tracking the pressure consistency which may be the average brushing pressure divided by the suggested pressure. This may identify whether the patient is applied too much, too little, or the correct amount of pressure overall or at a particular location.

A technique score may be provided based on the amount of time or area brushed with the techniques and settings provided in the guidance.

A coverage score may be provided that is based on the area covered by brushing and the total oral area that should have been brushed. Coverage less than 100% may indicate that the patient should pay more attention to where they are brushing.

A brushing time optimization or deviation score may be a difference between the actual brushing time and the recommended brushing time. The score may aid in providing feedback regarding whether the user is over or under brushing.

A consistency index may be the sum the average of the session performance metrics normalized on a scale of 0 to 1 with 0 being not met and 1 being met and between 0 and 1 indicating a degree to which the metrics were met where if all the metrics, divided by the number of sessions. An index of 1 indicating that that all metrics were met for each session and 0 indicating that no metrics were met of all sessions.

A risk of plaque retention score may be a weighted combination of low coverage (such as the inverse of the total brushing coverage), the time spent at an incorrect pressure, and time spent with poor technique. The higher the score may indicate a high risk of plaque retention and buildup.

At block 830, oral health issues may be identified. For example, the image data of the patient's oral cavity may be analyzed to identify locations of oral health problems. The system may detect dental defects, such as tooth decay, plaque buildup and/or dental caries (cavities), such as by using a plaque or caries stain before scanning the teeth or other dental and oral health issues. For example, the 2D images captured with the intraoral scanner of stained teeth. For example, a caries dye or disclosing solutions may have been applied to the teeth before or during the imaging process. The teeth may have been imaged with the intraoral scanner. The areas of decay absorb the stain and appear differently colored compared to the healthy enamel. For instance, common colors for these stains is red or blue, which provides a sharp contrast to the natural color of the tooth, making the carious lesions more visible. The 3D scan may capture 3D and color data for the teeth with the dyed or stained locations on the teeth indicating potential locations of caries. The system may identify locations of caries based on the location of the dye on the teeth.

Similar to detection of caries, stains may also be used to detect dental plague. A dye or stain that that selectively adheres to plaque, with little to no adherence to the clean surfaces of teeth may have been applied to the teeth before or during imaging. The dye binds to the plaque deposits, staining them a vivid color (commonly pink, red, or blue) that contrasts sharply with the natural color of teeth, making the plaque clearly visible. The areas of with plaque buildup absorb the stain and appear differently colored compared to the cleaned portions of the teeth which provides a sharp contrast to the natural color of the tooth, making the plaque more visible. The imaging may capture color data for the teeth with the dyed or stained locations on the teeth indicating potential locations of dental plaque buildup. The system may identify locations of caries based on the location of the dye on the teeth.

Subsurface defects may be detected using, for example near infrared imaging that penetrates into the volume of the teeth to reveal subsurface structures, such as the enamel and its thickness, the dentin location and its thickness, and dental caries. Tooth decay, wear, and/or damage may be determined based on changes to the patient's teeth over time, such as by detecting changes in the external shape of teeth across multiple 3D scans gathered over a period of time, such as once a year, for multiple years.

The 3D scan may also reveal inflammation of the gingiva due to disease, surgery, dental implants, etc. Healthy gingiva, or gum tissue, typically presents as firm, pale pink, and tightly adhered to the underlying bone and teeth. It may have a smooth texture with a stippled appearance, often likened to the surface of an orange. The edges of healthy gingiva fit snugly around each tooth, forming a protective barrier that helps prevent the invasion of bacteria.

In contrast, inflamed gingiva often appears red or even purplish, swollen, and tends to bleed easily, particularly during brushing or flossing. The gingiva may also exhibit signs of formation of deep pockets between the teeth and gums. This condition results from the accumulation of bacterial plaque, which triggers an immune response leading to these characteristic changes.

The 3D scanner may employ structured light or other capture technology to capture the topography of the oral cavity. When scanning the oral cavity, including the gingiva, the scanner generates a precise 3D image if the gingiva. During the scanning process the 3D scanner may also capture color and textural data of the gingiva. The system may compare the captured 3D, color, and textural data to previous scans of the patient to determine if the patient's gingiva is inflamed. The system may analyze the captured 3D, color, and textural data to detect the presence red or purplish, swollen, gingiva and/or the formation of deep pockets between the teeth and gums to determine if the patient's gingiva is inflamed.

At block 840 a correlation may be made between toothbrushing data and oral health problems. For example, locations of gum recession that correspond to location of excess or high pressure while brushing or excess time brushing may indicate that the gum recession may be address by decreasing pressure or brushing time at those location while gum recession locations that correlate with lack of adequate bruising may indicate that the gum recession is caused by inadequate brushing or incorrect brushing techniques.

At block 850 guidance may be provided to improve the patient's oral health. For example, for gum recession the dentist may provide guidance regarding specific brushing techniques (gentle brushing mode) to reduce pressure and avoid further gum damage. Dentists can and/or the system may create a tailored treatment plan, which may include professional cleanings, scaling and root planning, or surgical interventions, based on the severity and progression of the recession. For plaque buildup patients can receive targeted guidance on how to improve brushing coverage, use of water flossers, and potentially schedule more frequent professional cleanings. For cavities and/or tooth decay dentists and/or the system may provide specific guidance for improving oral hygiene, such as using fluoride toothpaste, focusing on trouble spots (hard-to-reach areas), or reducing sugary foods and drinks. For inflammation around dental implants or surgical sites dentists may provide guidance regarding cleaning techniques, recommend special tools for implant care, and schedule regular check-ups to ensure the implant remains healthy. For crowded teeth patients can track coverage using smart toothbrush data. Dentists and/or the system may also provide guidance on using specific tools like angled brushes or flossers to clean these difficult areas, reducing the risk of decay and gum disease.

A dentist and/or the system may also provide proactive identification of potential issues based on the brushing history and imaging. For example, the system may detect problematic brushing habits such as based on detection of aggressive brushing and/or neglecting certain areas, warn patients about potential issues, such as based on enamel erosion, gum recession detected in the imaging, and educate patients and/or provide guidance on proper techniques to proactively prevent problems based on observed brushing techniques.

In some embodiments, the methods herein, such as methods 600, 700, and 800 may aid in visualizing areas with consistent plaque and tartar build-up based on smart toothbrush data, highlighting regions where brushing is inadequate, correlating with scanner images showing plaque, identifying excessive wear on specific teeth, correlating with aggressive brushing habits, and providing insights into potential issues like enamel erosion or receding gums.

In some embodiments, the methods herein, such as methods 600, 700, and 800 may aid in monitoring compliance with tooth brushing guidance and best practices. Smart toothbrushes may be used to track brushing the frequency and duration and other aspects of tooth brushing, the system can monitor if the patient consistently follows oral hygiene routines during treatment, and adjustments to the treatment plans or guidance can be provided by the doctor or the system to the patient if compliance is lacking from the patient.

For example, a method may be implemented using a smart toothbrush, a patient smart device such as a smartphone, and optionally one or more back-end computer systems associated with a dental practice or service provider.

The method may include providing tooth brushing guidance to a patient. The guidance may be general guidance based on dental best practices, treatment-specific guidance associated with an ongoing dental procedure, or patient-specific guidance, as discussed herein. The guidance may include recommended brushing frequency, brushing duration, brushing schedules, and other brushing-related parameters, as discussed herein.

During use by the patient, the smart toothbrush captures brushing data associated with one or more brushing sessions. The brushing data may include, for example, brushing frequency, brushing duration, and timing of brushing events, and other data, as discussed herein. In some embodiments, the brushing data further includes additional aspects of tooth brushing, such as operational states of the toothbrush or user interaction patterns.

In some embodiments, a smart toothbrush communicates the captured brushing data to a smart device associated with the patient. The smart device may store the brushing data locally and or transmit the brushing data to a back-end computer system. In some embodiments, the back-end computer system aggregates brushing data across multiple brushing sessions and maintains a brushing history for the patient. In some embodiments, the smartphone aggregates brushing data across multiple brushing sessions and maintains a brushing history for the patient.

The method may include monitoring the brushing data and determining whether the patient is complying with the tooth brushing guidance. Monitoring may include comparing the brushing frequency, brushing duration, or other captured aspects of brushing against expected values defined by the guidance. Based on this comparison, the system determines whether the patient consistently follows recommended oral hygiene routines during treatment.

If the monitoring indicates that compliance is lacking, the method may include generating one or more compliance-related outputs. The outputs may include notifications, reminders, or feedback presented to the patient via the smart device to encourage improved brushing behavior. In some embodiments, the system adjusts the tooth brushing guidance provided to the patient, such as modifying recommended brushing routines or emphasizing particular aspects of brushing, such as by providing additional information and guidance.

In some embodiments, compliance information is made available to a dental professional. The dental professional may review the compliance information and, based on the information, adjust a treatment plan, provide updated guidance, or communicate additional instructions to the patient. In some embodiments, adjustments to treatment plans, additional instructions, and/or guidance are generated automatically by the system based on detected compliance patterns.

In some embodiments, the methods herein, such as methods 600, 700, and 800 may aid in evaluating a patient during a dental checkup. The brushing patterns may be analyzed to identify areas of the teeth that the patient may miss during brushing or that they may not brush properly. Dentists can focus on those specific regions during check-ups and address missed locations to aid in improving overall oral health.

In some embodiments, the method may be performed by one or more of a smart toothbrush, a smart device associated with the patient, and one or more back-end computing systems.

The method may include receiving brushing data generated by a smart toothbrush during one or more brushing sessions performed by the patient, as discussed herein. The brushing data may include data indicative of brushing motion, brushing duration, brushing frequency, or other brushing-related activity, as discussed herein. The method further includes analyzing the brushing data across multiple brushing sessions to determine brushing patterns associated with the patient, as discussed herein.

Based on the determined brushing patterns, the method may include identifying one or more tooth regions that are potentially missed or inadequately brushed. Identification of such regions may be based on a determination that brushing activity associated with a particular tooth region fails to satisfy one or more expected brushing parameters, such as minimum brushing duration, frequency, or consistency across sessions, as discussed herein.

The method further includes generating region-based brushing information identifying the one or more tooth regions that are potentially missed or inadequately brushed and providing this information to a dental professional or a dental practice system. During a dental check-up, the dental professional may focus on the identified tooth regions, including examining the regions, addressing oral health conditions associated with the regions, and providing targeted oral hygiene instruction to the patient.

In some embodiments, the method may include updating tooth brushing guidance provided to the patient based on the identified tooth regions, input from the dental professional, or both. The updated guidance may be communicated to the patient via the smart device.

In some embodiments, the methods herein, such as methods 600, 700, and 800 may aid in providing updated guidance to a patient without a dental checkup. For example, the methods may monitor changes in brushing patterns over time to detect deviations, such as a decrease in brushing frequency or other aspect of toothbrushing described herein and then alert patients and dentists about the potential issues, such as gum recession, plague buildup, gum disease, etc, based on the observed brushing. In some embodiments, the method may provide insights such as and observed specific prolonged brushing pattern can lead to specific dental issue. The method may be performed by one or more of a smart toothbrush, a smart device associated with the patient, and one or more back-end computer systems.

The method may include receiving brushing data generated by a smart toothbrush during multiple brushing sessions performed by the patient over a period of time, as discussed herein. The brushing data may include brushing frequency, brushing duration, timing of brushing sessions, and other aspects of tooth brushing described herein. The method further includes analyzing the brushing data to establish one or more baseline brushing patterns associated with the patient, as discussed herein.

The method may include monitoring the brushing data over time to detect deviations from the baseline brushing patterns. The detected deviations may include, for example, a decrease in brushing frequency, a reduction in brushing duration, changes in brushing consistency, or other deviations in brushing behavior. In some embodiments, the deviations are identified by comparing recent brushing data to historical brushing data or expected brushing parameters.

Based on the detected deviations, the method may include identifying one or more potential oral health issues associated with the observed brushing behavior. The potential oral health issues may include, for example, gum recession, plaque buildup, gingival inflammation, gum disease, or other oral health conditions. In some embodiments, the identification is based on one or more correlations between specific brushing patterns or prolonged brushing behaviors and known dental issues.

The method may include generating alerts or notifications regarding the detected deviations and the potential oral health issues. The alerts may be provided to the patient via the smart device and or to a dental professional via a dental practice system, such as those discussed herein. The alerts may include information describing the detected brushing deviations, associated risks, and recommended actions, such as those discussed herein.

In some embodiments, the method may inlcude generating insights relating brushing patterns to oral health outcomes. The insights may indicate that a specific prolonged brushing pattern, or a specific change in brushing behavior over time, is associated with an increased likelihood of a particular dental issue. The insights may be used to update tooth brushing guidance, inform clinical decision-making, or educate the patient regarding improved oral hygiene practices.

In some embodiments, customized brushing treatment plans may be generated based on methods 600, 700, and 800 including guidance on specific products, such as tooth pasted, mouthwash, toothbrushes, and toothbrushing and oral health care techniques. Plans may also include treatments for breath odor, plaque buildup, tooth whitening, gum health, care of aligners and/or braces or other oral appliances. If improvements in these areas are not met, they may seek assistance from a dental professional.

In some embodiments, a method may include receiving brushing data generated by a smart toothbrush during one or more brushing sessions performed by the patient. The brushing data may include brushing frequency, brushing duration, timing of brushing sessions, and other aspects of toothbrushing described herein. The method further includes receiving oral health data associated with the patient. The oral health data may include, for example, clinician-entered observations, patient-reported symptoms, dental images or scans, dental records, and/or other data indicative of oral health status and treatment objectives.

The method may include analyzing the brushing data and the oral health data to determine one or more oral care needs, risks, or objectives for the patient. In some embodiments, the analysis includes identifying patterns in brushing behavior, such as under-brushing, inconsistent brushing routines, prolonged high-force brushing, or other deviations described herein and correlating the patterns with potential oral health concerns, such as plaque buildup risk, gum irritation risk, breath odor risk, aligner or brace care needs, or whitening goals. In some embodiments, the analysis includes determining one or more target outcomes, such as improved brushing consistency, improved gum health, reduced plaque buildup, improved breath odor, tooth whitening progress, or improved care of aligners, braces, or other oral appliances.

Based on the analysis, the method may include generating a customized brushing and oral care treatment plan for the patient. The customized treatment plan may include personalized guidance for toothbrushing routines and oral hygiene best practices, including recommended brushing frequency, duration, and technique-related guidance. In some embodiments, the customized treatment plan includes recommendations for specific oral care products tailored to the patient's needs, such as a particular toothpaste, mouthwash, or toothbrush type. In some embodiments, the plan includes guidance on oral health care techniques beyond toothbrushing, such as rinsing routines, tongue cleaning, flossing or interdental cleaning, appliance-cleaning routines for aligners and or braces, or other oral care behaviors.

In some embodiments, the customized treatment plan includes a plurality of plan modules directed to respective oral health objectives or conditions. The plan modules may include, for example, a breath odor module, a plaque buildup module, a tooth whitening module, a gum health module, and an aligner or brace care module. Each module may specify one or more recommended routines, product usage guidance, schedules, or technique recommendations associated with the module objective.

In some embodiments, the method includes providing the customized treatment plan to the patient via the smart device and storing the treatment plan in memory. The method further includes monitoring patient progress with respect to the customized treatment plan using subsequent brushing data and or subsequent oral health data. In some embodiments, monitoring includes determining whether one or more improvement metrics are met, such as increased brushing frequency or duration relative to a baseline, improved consistency of brushing routines, reduced deviations associated with high-risk brushing patterns, or other plan-specific progress indicators.

If the monitoring indicates that expected improvements associated with the customized treatment plan are not met, the method further includes generating an escalation output. The escalation output may prompt the patient, via the smart device, to seek assistance from a dental professional. In some embodiments, the method further includes providing a summary to the dental professional or a dental practice system that includes the customized treatment plan, observed brushing behavior, and one or more areas in which improvement has not been achieved, thereby enabling the dental professional to provide revised guidance, adjust the plan, or modify a dental treatment plan.

In some embodiments, if the monitoring indicates that expected improvements associated with the customized treatment plan are met, the method further includes generating an updated treatment plan based on the improved oral health status of the patient. The updated treatment plan may reflect reduced risk levels, achieved treatment objectives, or progression to maintenance-oriented care. In some embodiments, generating the updated treatment plan includes modifying one or more aspects of the existing plan, such as adjusting recommended brushing frequency or duration, reducing emphasis on previously high-risk oral health objectives, or transitioning one or more plan modules from a corrective mode to a maintenance mode.

In some embodiments, the updated treatment plan includes revised guidance for oral care products, techniques, or routines that are appropriate for the patient's improved dental health. For example, the method may recommend continued use of certain products, techniques, or routines that contributed to improvement, introduce alternative products, techniques, or routines suited for long-term maintenance, or reduce the intensity or frequency of certain interventions. The updated treatment plan may also introduce new oral health objectives, such as cosmetic maintenance, long-term gum health preservation, or ongoing care of aligners, braces, or other oral appliances.

The method may further include providing the updated treatment plan to the patient via the smart device and storing the updated treatment plan in memory. In some embodiments, the updated treatment plan is also made available to a dental professional or a dental practice system.

The system and methods herein may also be used to aid in increasing patient compliance. For example, through gamification of the best practices, prescriptions, and guidance by setting milestone to raise awareness of the patient's brushing techniques and consistency. Challenges may be set based on the brushing guidelines described herein and patients may be rewarded with points, badges, or virtual prizes for achieving goals via feedback on their smart computing device.

In some embodiments, a method may increase patient compliance with tooth brushing guidance through gamification. The method may include presenting brushing goals to a patient via a smart device, where the brushing goals are derived from brushing guidelines, prescriptions, or guidance described herein. The brushing goals may include targets related to brushing frequency, brushing duration, brushing consistency over time, or adherence to recommended brushing routines, or other goals. The method may include monitoring brushing data generated by a smart toothbrush, as discussed herein, to determine patient progress toward the brushing goals. As the patient completes brushing sessions that satisfy the brushing goals, the method may include awarding virtual rewards, such as points, badges, or virtual prizes, and presenting feedback on the smart device to reinforce compliant brushing behavior.

In some embodiments, a method may increase patient compliance with milestone-based gamification of tooth brushing techniques. The method may include defining one or more, such as a plurality, of milestones associated with brushing, where each milestone corresponds to achieving a specific brushing technique objective or consistency objective. The milestones may include, for example, brushing for a prescribed duration for a defined number of consecutive days, achieving a target brushing frequency over a week, or maintaining compliant brushing behavior for specific tooth regions, as discussed herein. The method may include tracking brushing data over time to determine when a milestone is achieved and, in response, generating a milestone achievement notification and awarding a corresponding virtual reward to the patient via the smart device.

In some embodiments, challenge-based gamification may be used to improve brushing compliance. A method may include generating one or more brushing challenges for the patient based on brushing guidelines described herein. The challenges may be time-limited challenges, region-specific challenges, technique-focused challenges, or consistency challenges.

In some embodiments, brushing challenges are generated based on tooth brushing guidance to encourage patient compliance through structured, goal-oriented activities. The challenges may take several forms, including time-limited challenges, region-specific challenges, technique-focused challenges, and consistency challenges, each based on recommended brushing practices, as discussed herein.

In some embodiments, a time-limited challenge may be defined by a specified duration during which the patient is encouraged to satisfy one or more brushing guidance parameters. For example, a time-limited challenge may require the patient to complete brushing sessions that meet a recommended brushing duration and/or frequency within a defined period, such as completing at least two brushing sessions per day for a consecutive number of days.

In some embodiments, a region-specific challenge may be defined by brushing guidance associated with one or more specific tooth regions. The region-specific challenge may encourage the patient to adequately brush designated areas of the dentition, such as posterior teeth, lingual surfaces, or other regions as provided by the guidance. The challenge may require that brushing activity associated with the specified regions meets one or more guidance parameters, such as minimum brushing duration or consistent coverage across multiple brushing sessions.

In some embodiments, a technique-focused challenge is defined by brushing guidance related to brushing technique. The technique-focused challenge may encourage adherence to recommended brushing techniques, such as maintaining a target brushing angle relative to the gum line, applying brushing force within a specified range, or performing a recommended brushing motion. The challenge may be satisfied when brushing data indicates that the patient consistently applies the recommended technique during one or more brushing sessions.

In some embodiments, a consistency challenge is defined by brushing guidance related to long-term brushing behavior. The consistency challenge may encourage the patient to maintain compliant brushing behavior across multiple brushing sessions over time. For example, a consistency challenge may require that the patient meets recommended brushing frequency and duration targets for a defined percentage of brushing sessions over a week or month.

The method further includes monitoring brushing activity during the challenge period and determining whether the challenge criteria are satisfied. Upon successful completion of a challenge, the method includes providing positive feedback and awarding virtual rewards. If the challenge criteria are not satisfied, the method may provide encouragement or modified challenges to maintain patient engagement.

In some embodiments, a method may include adaptive gamification based on patient brushing behavior. The method may include analyzing brushing data to determine a patient's current compliance level and engagement with brushing guidance. Based on the analysis, the method may include adjusting gamification parameters, such as difficulty level of challenges, milestone thresholds, or types of rewards, to better match the patient's behavior. For example, simpler challenges, such as lower time requirements, relaxed position or angle requirements of the brush to allow for greater deviations from desired angles, positions, or pressure may be provided to patients with low compliance, while more advanced challenges, such as with higher time requirements, and tighter position, angle, and brush pressure requirements relative to the guidance may be provided to patients demonstrating consistent compliance. The method further includes presenting the adapted challenges and rewards via the smart device to encourage continued participation.

In some embodiments, social or comparative gamification of brushing behavior may be used. The method may include presenting comparative feedback to the patient via the smart device, such as anonymized comparisons to average brushing behavior or personal past performance. In some embodiments, the method includes enabling optional participation in group challenges or shared milestones among a defined group of users, such as family members. Rewards may be generated based on individual or group performance, to leverage social motivation to increase compliance.

In some embodiments, a method is provided for dentist-guided gamification of brushing compliance. The method includes receiving gamification parameters from a dental professional or a dental practice system, such as customized goals, rewards, or challenge types aligned with a patient's treatment plan. The method further includes implementing the gamification using brushing data and presenting progress and rewards to the patient via the smart device. In some embodiments, gamification outcomes are made visible to the dental professional to support reinforcement of positive behavior during dental visits.

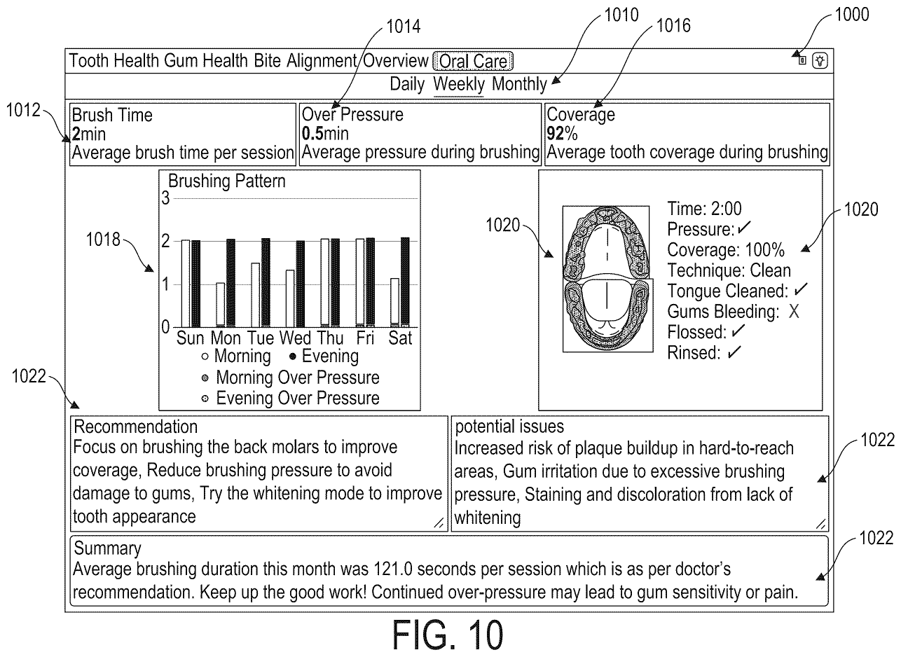

FIG. 10 depicts a summary 1000 of brushing data that may be generated by the system using the methods described herein. The summary may include daily, weekly, and/or monthly tabs 1010 for selecting a timeline to view the data. Each tab provides an average brush time 1012, average over-pressure applied during brushing 1014, and coverage (%) 1016 for the selected timeframe. Each tab may also include a graphical representation 1018, such as a bar graph of brush time & over-pressure for the selected period to see brushing pattern and coverage diagram 1020 highlighting what areas are missed/not properly covered while brushing. The tabs may also include a summary 1020 of brushing information, as discussed herein.

Editable text fields may include treatment recommendations and/or guidance from a doctor and the problems that may arise with current brushing patterns and behaviors along with a summary on patient's brushing patterns. The summary may be AI generated and confirmed by a dentist.

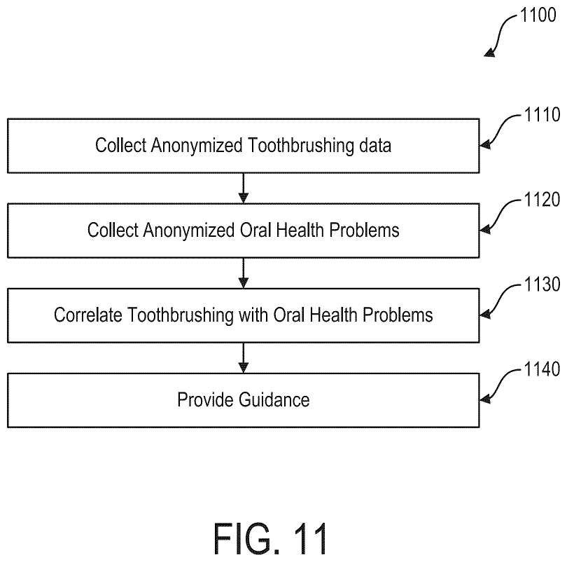

FIG. 11 depicts a method 1100 of data mining toothbrushing and oral health data to correlate toothbrushing techniques and patterns with oral health problems and to generate new and novel toothbrushing guidance based on the correlation. In the methods discussed above, correlations are made on a single patient to determine whether an oral health problem may be linked to improper brushing. In method 1100, the toothbrushing and oral identified oral health problems data is mined to better understand what types of tooth brushing techniques lead to or prevent oral health problems.

At block 1110 anonymized tooth brushing data is collected from many patients, such as hundreds, thousands, or even millions of patients. This tooth brushing data may be any or all of the data related to tooth brushing discussed herein.

At block 1120 anonymized oral health data is collected from many patients, such as hundreds, thousands, or even millions of patients. This oral health data may be any or all of the oral health data discussed herein.

At block 1130, the toothbrushing data is correlated with the oral health problems, such as through the use of artificial intelligence, data mining, or other data analysis methods. For example, it may be determined that brushing with the toothbrush angled at 30 degrees leads to improved plaque removal as compared to the currently recommended 45 degrees or that certain bristle types or arrangements lead to better cleaning of crowded teeth.

At block 1140, updated patient guidance on toothbrushing techniques may be generated and provided to doctors and patients.

As described herein, the computing devices and systems described and/or illustrated herein broadly represent any type or form of computing device or system capable of executing computer-readable instructions, such as those contained within the modules described herein. In their most basic configuration, these computing device(s) may each comprise at least one memory device and at least one physical processor. A computer-readable medium, such as a non-transitory computer readable medium, that may store instructions that, when executed by a computer perform any of the methods or processes described herein.

The term “memory” or “memory device,” as used herein, generally represents any type or form of volatile or non-volatile storage device or medium capable of storing data and/or computer-readable instructions. In one example, a memory device may store, load, and/or maintain one or more of the modules described herein. Examples of memory devices comprise, without limitation, Random Access Memory (RAM), Read Only Memory (ROM), flash memory, Hard Disk Drives (HDDs), Solid-State Drives (SSDs), optical disk drives, caches, variations or combinations of one or more of the same, or any other suitable storage memory.

In addition, the term “processor” or “physical processor,” as used herein, generally refers to any type or form of hardware-implemented processing unit capable of interpreting and/or executing computer-readable instructions. In one example, a physical processor may access and/or modify one or more modules stored in the above-described memory device. Examples of physical processors comprise, without limitation, microprocessors, microcontrollers, Central Processing Units (CPUs), Field-Programmable Gate Arrays (FPGAs) that implement softcore processors, Application-Specific Integrated Circuits (ASICs), portions of one or more of the same, variations or combinations of one or more of the same, or any other suitable physical processor. The processor may comprise a distributed processor system, e.g. running parallel processors, or a remote processor such as a server, and combinations thereof.

Although illustrated as separate elements, the method steps described and/or illustrated herein may represent portions of a single application. In addition, in some embodiments one or more of these steps may represent or correspond to one or more software applications or programs that, when executed by a computing device, may cause the computing device to perform one or more tasks, such as the method step.

In addition, one or more of the devices described herein may transform data, physical devices, and/or representations of physical devices from one form to another. Additionally or alternatively, one or more of the modules recited herein may transform a processor, volatile memory, non-volatile memory, and/or any other portion of a physical computing device from one form of computing device to another form of computing device by executing on the computing device, storing data on the computing device, and/or otherwise interacting with the computing device.

The term “computer-readable medium,” as used herein, generally refers to any form of device, carrier, or medium capable of storing or carrying computer-readable instructions. Examples of computer-readable media comprise, without limitation, transmission-type media, such as carrier waves, and non-transitory-type media, such as magnetic-storage media (e.g., hard disk drives, tape drives, and floppy disks), optical-storage media (e.g., Compact Disks (CDs), Digital Video Disks (DVDs), and BLU-RAY disks), electronic-storage media (e.g., solid-state drives and flash media), and other distribution systems.

A person of ordinary skill in the art will recognize that any process or method disclosed herein can be modified in many ways. The process parameters and sequence of the steps described and/or illustrated herein are given by way of example only and can be varied as desired. For example, while the steps illustrated and/or described herein may be shown or discussed in a particular order, these steps do not necessarily need to be performed in the order illustrated or discussed.

The various exemplary methods described and/or illustrated herein may also omit one or more of the steps described or illustrated herein or comprise additional steps in addition to those disclosed. Further, a step of any method as disclosed herein can be combined with any one or more steps of any other method as disclosed herein.

The processor as described herein can be configured to perform one or more steps of any method disclosed herein. Alternatively or in combination, the processor can be configured to combine one or more steps of one or more methods as disclosed herein.

Unless otherwise noted, the terms “connected to” and “coupled to” (and their derivatives), as used in the specification and claims, are to be construed as permitting both direct and indirect (i.e., via other elements or components) connection. In addition, the terms “a” or “an,” as used in the specification and claims, are to be construed as meaning “at least one of.” Finally, for ease of use, the terms “including” and “having” (and their derivatives), as used in the specification and claims, are interchangeable with and shall have the same meaning as the word “comprising.

The processor as disclosed herein can be configured with instructions to perform any one or more steps of any method as disclosed herein.

It will be understood that although the terms “first,” “second,” “third”, etc. may be used herein to describe various layers, elements, components, regions or sections without referring to any particular order or sequence of events. These terms are merely used to distinguish one layer, element, component, region or section from another layer, element, component, region or section. A first layer, element, component, region or section as described herein could be referred to as a second layer, element, component, region or section without departing from the teachings of the present disclosure.

As used herein, the term “or” is used inclusively to refer items in the alternative and in combination.

As used herein, characters such as numerals refer to like elements.

The present disclosure includes the following numbered clauses.

Clause 1. A method for using intraoral scan data to guide patient toothbrushing, the method comprising: receiving brushing guidance and a 3D model of a patient's dentition; activating the toothbrush to start a tooth brushing activity; determining, during toothbrushing activity, a location of the toothbrush with respect to the patient's dentition based on data from sensors on the toothbrush and the 3D model; correlating the toothbrush location with the brushing guidance; and adjusting an operation of the toothbrush based on the correlation of the toothbrush location with the brushing guidance.

Clause 2. The method of clause 1, wherein the sensors are image sensors and the data from the sensors is 2D image data.

Clause 3. The method of clause 2, wherein determining the location of the toothbrush with respect to the patient's dentition includes determining a location of an image sensor with respect to the patient's dentition by aligning the 2D image data with the 3D model.

Clause 4. The method of clause 3, wherein adjusting an operation of the toothbrush includes slowing a speed of an oscillation of the toothbrush head.

Clause 5. The method of clause 1, further comprising: providing feedback to a user of the toothbrush based on the correlation of the toothbrush location with the brushing guidance.

Clause 6. The method of clause 5, wherein the feedback is guidance to adjust a position of the toothbrush.

Clause 7. The method of clause 1, further comprising: determining, during toothbrushing activity, an orientation of the toothbrush with respect to the patient's dentition based data from sensors on the toothbrush and the 3D model.

Clause 8. The method of clause 7, wherein the feedback is guidance to adjust a position of the toothbrush.

Clause 9. A method for using intraoral scan data to improve oral health, the method comprising: receiving first scan data of a patient's dentition at a first time; receiving second scan data of a patient's dentition at a second time; receiving toothbrushing tracking data of a patient's toothbrushing for a plurality of toothbrushing sessions between the first time and the second time; identifying from the first scan data and the second scan data types and locations of oral health changes of the patient's dentition between the first scan data and the second scan data; and providing toothbrushing guidance based on the type and location of the oral health changes.

Clause 10. The method of clause 9, wherein the guidance is an orientation of the toothbrush at a location of the oral health changes.

Clause 11. The method of clause 9, wherein the toothbrush tracking data includes toothbrushing time mapped to dentition location.

Clause 12. The method of clause 9, wherein the toothbrush tracking data includes toothbrushing pressure mapped to dentition location.

Clause 13. The method of clause 12, wherein the oral health problems include gum recession.

Clause 14. The method of clause 12, wherein the oral health problems include plaque buildup.

Clause 15. The method of clause 9, further comprising: generating toothbrushing tracking data by determining, during the plurality of toothbrushing sessions, the location of the toothbrush with respect to the patient's dentition based on data from sensors on the toothbrush and the 3D model.

Clause 16. A system for using intraoral scan data to guide patient toothbrushing, the system comprising: an electronic toothbrush including: a toothbrush head having bristles and being configured to oscillate; one or more sensors configured to record sensor data for determining the position and orientation of the toothbrush relative to the patient's dentition; a first processor configured to control the operation of the electronic toothbrush; a smart device including configured to receive and store brushing guidance, a 3D model of a patient's dentition, and the sensor data, the smart device including: a second processor configured to: determine, during toothbrushing activity, a location of the toothbrush head with respect to the patient's dentition based on the sensor data and the 3D model; correlate the toothbrush location with the brushing guidance; and send an instruction to the electronic toothbrush to adjust an operation of the toothbrush based on the correlation of the toothbrush location with the brushing guidance.

Clause 17. The system of clause 16, wherein the sensors are image sensors and the data from the sensors is 2D image data.

Clause 18. The system of clause 17, wherein the one or more sensors includes an image sensor and determining the location of the toothbrush with respect to the patient's dentition includes determining a location of an image sensor with respect to the patient's dentition by aligning the 2D image data with the 3D model.

Clause 19. The system of clause 18, wherein adjusting an operation of the toothbrush includes slowing a speed of an oscillation of the toothbrush head.

Clause 20. The system of clause 16, wherein the second processor is further configured to provide feedback to a user of the electronic toothbrush based on the correlation of the toothbrush location with the brushing guidance.

Clause 21. The system of clause 20, wherein the feedback is guidance to adjust a position of the toothbrush.

Clause 22. The system of clause 16, wherein the second processor is further configured to determine, during toothbrushing activity, an orientation of the toothbrush with respect to the patient's dentition based data from sensors on the toothbrush and the 3D model.

Clause 23. The system of clause 22, wherein the feedback is guidance to adjust a position of the toothbrush.

Embodiments of the present disclosure have been shown and described as set forth herein and are provided by way of example only. One of ordinary skill in the art will recognize numerous adaptations, changes, variations and substitutions without departing from the scope of the present disclosure. Several alternatives and combinations of the embodiments disclosed herein may be utilized without departing from the scope of the present disclosure and the inventions disclosed herein. Therefore, the scope of the presently disclosed inventions shall be defined solely by the scope of the appended claims and the equivalents thereof.

What is claimed is:

1. A method for using intraoral scan data to guide patient toothbrushing, the method comprising:
receiving brushing guidance and a 3D model of a patient's dentition;
determining, during toothbrushing activity, a location of the toothbrush with respect to the patient's dentition based on data from sensors on the toothbrush and the 3D model;
correlating the toothbrush location with the brushing guidance; and
adjusting an operation of the toothbrush based on the correlation of the toothbrush location with the brushing guidance.

2. The method of claim 1, wherein the sensors are image sensors and the data from the sensors is 2D image data.

3. The method of claim 2, wherein determining the location of the toothbrush with respect to the patient's dentition includes determining a location of an image sensor with respect to the patient's dentition by aligning the 2D image data with the 3D model.

4. The method of claim 3, wherein adjusting an operation of the toothbrush includes slowing a speed of an oscillation of the toothbrush head.

5. The method of claim 1, further comprising:
providing feedback to a user of the toothbrush based on the correlation of the toothbrush location with the brushing guidance.

6. The method of claim 5, wherein the feedback is guidance to adjust a position of the toothbrush.

7. The method of claim 1, further comprising:
determining, during toothbrushing activity, an orientation of the toothbrush with respect to the patient's dentition based data from sensors on the toothbrush and the 3D model.

8. The method of claim 7, wherein the feedback is guidance to adjust a position of the toothbrush.

9. A method for using intraoral scan data to improve oral health, the method comprising:
receiving first scan data of a patient's dentition at a first time;
receiving second scan data of a patient's dentition at a second time;
receiving toothbrushing tracking data of a patient's toothbrushing for a plurality of toothbrushing sessions between the first time and the second time;
identifying from the first scan data and the second scan data types and locations of oral health changes of the patient's dentition between the first scan data and the second scan data; and
providing toothbrushing guidance based on the type and location of the oral health changes.

10. The method of claim 9, wherein the guidance is an orientation of the toothbrush at a location of the oral health changes.

11. The method of claim 9, wherein the toothbrush tracking data includes toothbrushing time mapped to dentition location.

12. The method of claim 9, wherein the toothbrush tracking data includes toothbrushing pressure mapped to dentition location.

13. The method of claim 12, wherein the oral health problems include gum recession.

14. The method of claim 12, wherein the oral health problems include plaque buildup.

15. The method of claim 9, further comprising:
generating toothbrushing tracking data by determining, during the plurality of toothbrushing sessions, the location of the toothbrush with respect to the patient's dentition based on data from sensors on the toothbrush and the 3D model.

16. A system for using intraoral scan data to guide patient toothbrushing, the system comprising:
an electronic toothbrush including:
a toothbrush head having bristles and being configured to oscillate;
one or more sensors configured to record sensor data for determining the position and orientation of the toothbrush relative to the patient's dentition;
a first processor configured to control the operation of the electronic toothbrush;
instructions stored in memory that when executed by a second processor cause the second processor to:
receive and store brushing guidance, a 3D model of a patient's dentition, and the sensor data;
determine, during toothbrushing activity, a location of the toothbrush head with respect to the patient's dentition based on the sensor data and the 3D model;
correlate the toothbrush location with the brushing guidance; and
send an instructions to the electronic toothbrush to adjust an operation of the toothbrush based on the correlation of the toothbrush location with the brushing guidance.

17. The system of claim 16, wherein the sensors are image sensors and the data from the sensors is 2D image data.

18. The system of claim 17, wherein the one or more sensors includes an image sensor and determining the location of the toothbrush with respect to the patient's dentition includes determining a location of an image sensor with respect to the patient's dentition by aligning the 2D image data with the 3D model.

19. The system of claim 18, wherein adjusting an operation of the toothbrush includes slowing a speed of an oscillation of the toothbrush head.

20. The system of claim 16, wherein the second processor is further configured to provide feedback to a user of the electronic toothbrush based on the correlation of the toothbrush location with the brushing guidance.

21. The system of claim 20, wherein the feedback is guidance to adjust a position of the toothbrush.

22. The system of claim 16, wherein the second processor is further configured to determine, during toothbrushing activity, an orientation of the toothbrush with respect to the patient's dentition based data from sensors on the toothbrush and the 3D model.

23. The system of claim 22, wherein the feedback is guidance to adjust a position of the toothbrush.
